SEQUENCE LISTING (10 sequences)

SEQ ID NO: 1 - DNA, 23 bp, Artificial Sequence
  Primer
ttaagatcgaattggctcacgtt

SEQ ID NO: 2 - DNA, 23 bp, Artificial Sequence
  Primer
ttaagatcgaattggcccacgtt

SEQ ID NO: 3 - DNA, 13 bp, Artificial Sequence
  Primer
gatcgaattggcc

SEQ ID NO: 4 - DNA, 13 bp, Artificial Sequence
  Primer
gatcgaattggct

SEQ ID NO: 5 - DNA, 20 bp, Artificial Sequence
  Primer
cttctcctgcaggtgaccat

SEQ ID NO: 6 - DNA, 20 bp, Artificial Sequence
  Primer
cttctcctgcaggtgaccat

SEQ ID NO: 7 - DNA, 21 bp, Artificial Sequence
  Primer
ctgagggctgatccttcccag

SEQ ID NO: 8 - DNA, 19 bp, Artificial Sequence
  Primer
gagcatggcaggccctggc

SEQ ID NO: 9 - DNA, 22 bp, Artificial Sequence
  Primer
aatgagcatggcaggccctggt

SEQ ID NO: 10 - DNA, 21 bp, Artificial Sequence
  Primer
tcgaggcatttgccctgaaca

(19) United States
(12) Patent Application Publication
(10) Pub. No.: US 2002/0142336 A1
(43) Pub. Date: Oct. 3, 2002

(54) Methods for determining a nucleotide at a specific location within a nucleic acid molecule

(75) Inventors: Douglas R. Smith, Gloucester, MA (US); Hans-Ulrich Thomann, Lexington, MA (US); Patrick Cahill, Natick, MA (US)

Correspondence Address: LAHIVE & COCKFIELD, 28 STATE STREET, BOSTON, MA, 02109, US

(73) Assignee: Genome Therapeutics Corporation, Waltham, MA (US)

(21) Appl. No.: 10/061,961

(22) Filed: Feb. 1, 2002

Related U.S. Application Data
(60) Provisional application No. 60/266,035, filed on Feb. 2, 2001.

(51) Int. Cl. C12Q 1/68; C12P 19/34

(52) U.S. Cl. 435/6; 435/91.2

(57) ABSTRACT

Novel methods for determining the existence or nonexistence of a test nucleotide on a strand of DNA are provided. The methods involve the use of a proofreading polymerase that is capable of incorporating a labeled nucleotide in a primer into and extension product if there is a match between the test nucleotide on the strand of DNA and the complementary nucleotide on the primer, but which excises the labeled nucleotide and does not incorporate it into an extension product if there is a mismatch. The presence or absence of the test nucleotide then may be established by determining whether the label is preserved or lost following the reaction. Methods involving the use of a quencher-chromophore pair are also provided.

### RELATED APPLICATIONS

This application claims priority to U.S. Provisional Patent Application Serial No. 60/266,035 filed Feb. 2, 2001, the entire contents of which are incorporated herein by reference.

### BACKGROUND OF THE INVENTION

This invention relates to methods and products useful for detecting the presence or absence of a particular nucleotide at a specific location on a strand of DNA. By using the methods and products of this invention, it is possible to determine the genotype of an individual at any locus of interest.

A single nucleotide position on a strand of DNA may be responsible for a polymorphism or an allelic variation. There are known disease states that are caused by such variations at a single nucleotide position. The usefulness of detecting such variations includes but is not limited to gene typing, karyotyping, genotyping, DNA family planning, diagnostics (including infectious disease), prenatal testing, paternal determination, pharmacogenetics, and forensic analysis.

The typical methods used for determining such variations have been the use of Southern Blot analysis to test for variation in the length of specific DNA restriction fragments, or the use of a polymerase chain reaction (PCR) to amplify specific regions of DNA samples and test for nucleotide variation by sequence analysis (including single base extension, or “mini-sequencing”), or by hybridization with allele specific probes.

Each of these methods has certain drawbacks, including the lack of reproducibility of Southern analysis, the need for running gels to separate DNA fragments, and the extended amount of time required to complete the necessary steps in the process.

Standard PCR techniques suffer from the occurrence of false signals arising from contamination, and the time and technical expertise required for the determination of sequences from PCR amplified samples. However, perhaps the most serious drawback is that both methods require a number of separate analyses to test for variation at more than one DNA locus.

### SUMMARY OF THE INVENTION

The present invention involves a novel technique for determining the existence or nonexistence of a particular nucleotide at a specific locus on a strand of DNA, e.g., genomic DNA, at least a portion of which strand has a known sequence, adjacent to and including the locus of interest. The methods of the invention are particularly sensitive and rapid and have wide applications in various fields, including the field of pharmacogenomics.

Accordingly, the present invention provides a method for detecting the presence or absence of a first nucleotide, at a position within a DNA molecule in a sample (e.g., genomic DNA, a restriction endonuclease derived genomic or cDNA fragment, or a hydrodymanically or enzymatically sheared genomic or cDNA fragment) by forming an admixture of a primer and a strand of DNA of the sample and imposing conditions such that a hybridization product is formed between the primer and the DNA strand, the primer comprising a sequence of DNA which hybridizes with the strand of DNA adjacent to the first nucleotide position and having a second nucleotide opposite the first nucleotide position, the second nucleotide having an associated fluorescent label and hybridizing to the first nucleotide in the event the second nucleotide is complementary to the first nucleotide and the second nucleotide not hybridizing to the first nucleotide in the event of the second nucleotide is not complementary; applying a proofreading polymerase to the hybridization product under conditions in which the second nucleotide is preferentially excised to form a fluorescently labeled nucleotide excision product in the event the second nucleotide is not hybridized to the first nucleotide, and a fluorescently labeled primer extension product in the event that the second nucleotide is hybridized to the first nucleotide; and monitoring the sample for the presence of the fluorescent label in association with small molecules (the nucleotide excision product) versus large molecules (the primer extension product) using fluorescent polarization, the fluorescent label associated with an excess of small molecules being indicative of the absence of the first nucleotide, and the fluorescent label associated with an excess of large molecules being indicative of the presence of the first nucleotide.

The conditions for primer hybridization, nucleotide excision and primer extension may be carried out in the presence of a reverse primer in multiple cycles (e.g., during PCR amplification), or in an isothermal reaction in which new single-stand copies of the DNA molecules in the sample are faithfully generated, such as occurs in a rolling circle amplification (RCA) reaction where the strand of DNA of the sample that is being analyzed is a circular molecule, or has been circularized through some artificial means, e.g., by ligation of a linear fragment having blunt or cohesive ends derived from the sample, or by ligation of a “padlock probe” that is capable of hybridizing with the DNA molecule of the sample. Multiple primers differing at the position of the second nucleotide and having different labels associated therewith may also be used.

In another embodiment of the invention, the presence or absence of a label in the primer extension product is determined according to a method that requires an oligonucleotide primer having attached to it a unique tail sequence that is not complementary with the DNA of the sample. The primer extension product may then be applied to a substrate carrying an oligonucleotide, at least in part complementary to the unique tail sequence of the primer extension product, under conditions that allow the unique tail sequence to hybridize to the complementary oligonucleotide on the substrate. Preferably, both the tail and the oligonucleotide complementary to the tail are comprised of repeating units of complementarity. This favorably affects the kinetics of hybridization, increasing the speed and the sensitivity of the test. The complementary repeating units may consist of standard nucleic acid subunits capable of forming unique pairs, such as adenosine (A), thymidine (T) cytidine (C), or guanidine (G) nucleotides, or non-natural subunits such as peptide nucleic acid (PNA), ’iso-C', ’iso-G', ’Kappa', and Xanthosine.

In another aspect, the invention provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand, the primer comprising a sequence of DNA which hybridizes with the strand of DNA adjacent to the first nucleotide position and having a second nucleotide opposite the first nucleotide position, the second nucleotide having an associated mass-tag (e.g., an electrophore release tag) and hybridizing to the first nucleotide in the event that the second nucleotide is complementary to the first nucleotide and the second nucleotide not hybridizing to the first nucleotide in the event that the second nucleotide is not complementary; applying a proofreading polymerase to the hybridization product under conditions in which the second nucleotide is preferentially excised to form a mass-tag labeled nucleotide excision product in the event that the second nucleotide is not hybridized to the first nucleotide and a mass-tag labeled primer extension product in the event that the second nucleotide is hybridized to the first nucleotide; and monitoring the sample for the presence of the mass-tag in association with the nucleotide excision product, or the primer extension product using mass spectrometry (MS), the presence the mass-tag in association with the nucleotide excision product in concentrations greater than background being indicative of the absence of the first nucleotide, and the presence of the mass-tag in association with the primer extension product being indicative of the presence of the first nucleotide. As before, multiple rounds of excision and primer extension may be carried out by the application of PCR or isothermal amplification conditions (e.g., RCA). The use of hybridization tails to capture the extension product can be applied separately or in combination with any of these implementations of the invention. Multiple primers differing at the position of second nucleotide and having different mass-tags associated therewith may also be used. Different MS techniques (e.g., matrix-assisted laser desorption/ionization time-of-flight MS, laser-induced electron capture time-of-flight MS, gas chromatography electron capture MS, quadrapole MS, electrospray MS, liquid chromatography MS, Fourrier transform MS), may be used depending on the chemical properties of the mass-tags being detected.

In another aspect, the invention provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand, the primer comprising a sequence of DNA which hybridizes with the strand of DNA adjacent to the first nucleotide position and having a second nucleotide opposite the first nucleotide position, the second nucleotide having an associated label (eg., a fluorescent label, a mass-tag) and hybridizing to the first nucleotide in the event that the second nucleotide is complementary to the first nucleotide and the second nucleotide not hybridizing to the first nucleotide in the event that the second nucleotide is not complementary; applying a proofreading polymerase to the hybridization product under conditions in which the second nucleotide is preferentially excised to form a labeled nucleotide product in the event that the second nucleotide is not hybridized to the first nucleotide, and in which the second nucleotide is preferentially incorporated into a primer extension product in the event that the second nucleotide is hybridized to the first nucleotide; providing a dialysis chamber having a dialysis membrane (e.g., a semi-permeable polysulfone membrane or hollow microfiber) wherein the dialysis membrane is selected to have a molecular weight cut-off such that the labeled nucleotide excision product may pass through quickly, the primer may pass through slowly, and the extension product may not pass through; providing a means for introducing a sample into a chamber on the first side of the dialysis membrane (e.g., a syringe docking port and a vent hole each located near an opposite end of a dialysis chamber); introducing a dialysis solution into a chamber on the second side of the dialysis membrane opposite the first side of the dialysis membrane, and monitoring the sample on the first side and the dialysis solution on the second side for the presence of a label after providing sufficient time for dialysis of the various components in the sample to occur; the presence of a label in the dialysis solution in concentrations greater than background after a short time (nucleotide excision product) is indicative of the absence of the first nucleotide, and the presence of a label remaining in the sample chamber in concentrations greater that background after a longer time (extension product) is indicative of the presence of the first nucleotide. As before, multiple rounds of excision and primer extension may be carried out by the application of PCR or by the application of isothermal amplification conditions (e.g., RCA). Multiple primers differing at the position of second nucleotide and having different labels associated therewith may also be used.

In yet a further aspect, the invention provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand, the primer comprising a sequence of DNA which hybridizes with the strand of DNA adjacent to the first nucleotide position and having a second nucleotide opposite the first nucleotide position and hybridizing to the first nucleotide in the event that the second nucleotide is complementary to the first nucleotide and the second nucleotide not hybridizing to the first nucleotide in the event that the second nucleotide is not complementary; applying a proofreading polymerase to the hybridization product in the presence of a mixture of labeled dideoxynucleotides under conditions in which the second nucleotide is preferentially excised and a labeled dideoxunucleotide is added to the primer in the event that the second nucleotide is not hybridized to the first nucleotide, and in which the second nucleotide of the primer is preferentially extended with a labeled dideoxynucleotide in the event that the second nucleotide is hybridized to the first nucleotide; and monitoring the sample for the presence of a label in association with the primer, the nature of the label associated with the primer, and the length of the extension product being indicative of the identity of the first nucleotide.

In another aspect, the invention provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand, the primer comprising a sequence of DNA which hybridizes with the strand of DNA and having a second nucleotide containing a fluorescent label opposite the first nucleotide position and containing a quencher moiety attached at a position adjacent to the second nucleotide, the second nucleotide hybridizing to the first nucleotide in the event that the second nucleotide is complementary to the first nucleotide and the second nucleotide not hybridizing to the first nucleotide in the event that the second nucleotide is not complementary; applying a proofreading polymerase to the hybridization product under conditions in which the second nucleotide is preferentially excised in the event that the second nucleotide is not hybridized to the first nucleotide and in which the second nucleotide is preferentially incorporated into primer extension product in the event that the second nucleotide is hybridized to the first nucleotide; and monitoring the sample for emission from the fluorophore, the presence of fluorescence emission at levels greater than background being indicative of the absence of the first nucleotide, and the absence of fluorescence emission being indicative of the presence of the first nucleotide. As before, multiple rounds of excision and primer extension may be carried out by the application of PCR or isothermal amplification conditions (e.g., RCA). The use of hybridization tails to capture the extension product can be applied separately or in combination with any of these implementations of the invention. Multiple primers differing at the position of second nucleotide and having different fluorescent labels associated therewith may also be used. The quencher moiety is attached 1 -10 nucleotides away from the second nucleotide (containing the fluorescent label). Preferably, the quencher moiety is capable of quenching effectively over a broad wavelength range.

In another aspect, the invention provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand, the primer comprising a sequence of DNA which hybridizes with the strand of DNA and having a second nucleotide containing a quencher moiety opposite the first nucleotide position and containing a fluorescent label attached at a position adjacent to the second nucleotide, the second nucleotide hybridizing to the first nucleotide in the event that the second nucleotide is complementary to the first nucleotide and the second nucleotide not hybridizing to the first nucleotide in the event that the second nucleotide is not complementary; applying a proofreading polymerase to the hybridization product under conditions in which the second nucleotide is preferentially excised in the event that the second nucleotide is not hybridized to the first nucleotide and in which the second nucleotide is preferentially incorporated into primer extension product in the event that the second nucleotide is hybridized to the first nucleotide; and monitoring the sample for emission from the fluorophore, the presence of fluorescence emission at levels greater than background being indicative of the absence of the first nucleotide, and the absence of fluorescence emission being indicative of the presence of the first nucleotide. As before, multiple rounds of excision and primer extension may be carried out by the application of PCR or isothermal amplification conditions (e.g., RCA). The use of hybridization tails to capture the extension product can be applied separately or in combination with any of these implementations of the invention. Multiple primers differing at the position of second nucleotide and having different fluorescent labels associated therewith may also be used. The fluorescent label is attached about 1-10 nucleotides away from the second nucleotide (containing the quencher moiety). Preferably, the quencher moiety is capable of quenching effectively over a broad wavelength range.

In another aspect, the invention provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand, the primer comprising a sequence of DNA which hybridizes with the strand of DNA and having a second nucleotide containing an electrophoretic tag (e-tag) label opposite the first nucleotide position, the second nucleotide hybridizing to the first nucleotide in the event that the second nucleotide is complementary to the first nucleotide and the second nucleotide not hybridizing to the first nucleotide in the event that the second nucleotide is not complementary; applying a proofreading polymerase to the hybridization product under conditions in which the second nucleotide is preferentially excised in the event that the second nucleotide is not hybridized to the first nucleotide and in which the second nucleotide is preferentially incorporated into primer extension product in the event that the second nucleotide is hybridized to the first nucleotide; and monitoring the sample for the presence of free e-tag labeled nucleotide products at levels greater than background by electrophoretic separation, the presence of such labeled nucleotides being indicative of the absence of the first nucleotide, and the absence of such products being indicative of the presence of the first nucleotide. As before, multiple rounds of excision and primer extension may be carried out by the application of PCR or isothermal amplification conditions (e.g., RCA). Multiple primers differing at the position of second nucleotide and having different e-tags associated therewith may also be used in a multiplex assay.

The present invention further provides kits containing the components of the methods of the invention. For example, the kits may include a plurality of different oligonucleotide primers and a plurality of oligonucleotides complementary to portions of DNA extended by the action of the polymerase/exonuclease on the primer-test DNA hybridization product. Preferably, the kit may include a substrate having attached thereto the complementary oligonucleotides.

Other features and advantages of the invention will be apparent from the following detailed description, and from the claims.

### BRIEF DESCRIPTION OF THE DRAWINGS

FIG. 1 schematically shows the synthesis of an extension product with retention of label obtained by using the proofreading polymerase of the invention when there is a match at the test position.

FIG. 2 schematically illustrates the synthesis of an extension product with loss of label obtained by using the proofreading polymerase of the invention when there is a mismatch at the test position.

FIG. 3 schematically illustrates the synthesis of PCR products using the proofreading polymerase of the invention and two primers with different labels on the 3′ nucleotide (L1 and L2) to test for the identity of a SNP present on two Alleles (Allele 1 having a C:G pair at the variable position, and Allele 2 having an A:T pair). A sample which is heterozygous for the two Alleles would produce all four of the PCR products shown, whereas a sample that is homozygous for one Allele would produce only the pair of products indicated.

FIG. 4 schematically shows an oligonucleotide of one embodiment of the invention, which primer includes a unique tail sequence.

FIG. 5 schematically shows the detection of an extension product formed with retention of label on the oligonucleotide primer of FIG. 3.

FIG. 6 schematically shows a detection substrate for detecting the label on the oligonucleotide primer of FIG. 3.

FIG. 7 schematically shows a set of preferred oligonucleotide primers.

FIG. 8 schematically shows a substrate for determining allelic variation.

FIG. 9 schematically shows a second set of oligonucleotide primers for detecting multiple alleles at multiple loci.

FIG. 10 is a schematic representation of the single nucleotide replacement assay.

FIGS. 11 and 12 are schematic representations of the quencher-fluorophore assay.

FIG. 13 shows the results of an exo-proofreading assay that was used for the detection of a single nucleotide polymorphism (SNP) at position 341 in the NAT-2 gene.

FIG. 14 shows the results of an exo-proofreading assay that was used to score a single nucleotide polymorphism in the ASM698₁₃B ₁₃ 1 gene using a mixture of two primers having different labels (each primer being specific for a particular allele).

FIG. 15 shows the results of an exo-proofreading assay that was used to determine a single nucleotide polymorphism (GTC216+Q1) directly from genomic DNA using a mixture of two primers having different labels (each primer being specific for a particular allele).

FIG. 16 shows results from an integrated dialysis experiment to detect fluorescent-labeled excised nucleotide and PCR extension products generated in the exo-proofreading SNP assay.

FIG. 17 shows the results of an exo-proofreading assay that was used to score a single nucleotide polymorphism (NAT2 475) in primary PCR products using a mixture of two primers having different labels and detection by fluorescence polarization.

FIG. 18 shows the results of an exo-proofreading assay that was used to score a single nucleotide polymorphism (NAT2 475) in primary PCR products using a primer labeled with an electrophore mass-tag and detection by electron capture mass spectrometry.

FIG. 19 is a graph depicting a controlled titration of samples containing various ratios of C and T at the SNP position within theAsm_{—}454 gene. Based on the graph, the relative frequency of the base within a pooled set of samples can be determined.

### DETAILED DESCRIPTION OF THE INVENTION

The present invention involves a novel technique for determining the existence or nonexistence of a particular nucleotide at a specific locus on a strand of DNA, at least a portion of which strand has a known sequence, adjacent to and including the locus of interest. The invention may be used in connection with many medical tests, including gene typing, karyotyping, genotyping, DNA family planning, diagnostics (including infectious disease), prenatal testing, paternal determination, and forensic analysis. It is particularly useful in determining an individual's genotype at the test locus, especially as the genotype relates to the existence of an allele or mutation responsible for a disease state a response to a particular therapeutic agent, or as it relates to an individual's identity.

A particularly useful application of the methods of the invention is in the field of pharmacogenomics. Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M.W. et al. (1997) Clin. Chem. 43(2):254-266. Pharmacogenomics is a particularly useful tool for the clinical development of drugs. Pharmacogenomics allows a clinician or physician to target prophylactic or therapeutic treatments to patients who will most benefit from the treatment and to avoid treatment of patients who will experience toxic drug-related side effects.

Using the methods of the present invention a high resolution map can be generated from a subset of some ten-million single nucleotide polymorphisms (SNPs) in the human genome. As used herein, an “SNP” is an alteration that occurs in a measurable percentage of the general population, or of a particular subgroup, affecting a single nucleotide base in a stretch of DNA. In the case of humans, SNPs occur at a frequency of about one per every 1000 bases of DNA, on average, when the genomic DNA of any two individuals is compared. SNPs may be involved in disease processes, however, the vast majority are not disease-associated. Given sufficient data based on a large number of SNP genotypes, individuals can be grouped into genetic categories depending on a particular pattern of SNPs, and SNP haplotypes, in their individual genome. In such a manner, disease associated genes may be identified, and treatment regimens can be tailored to groups of genetically similar individuals.

In one aspect, the present invention involves the use of one or more labeled oligonucleotide primers that will either create a base pair match or mismatch between a test nucleotide on a DNA strand and the labeled nucleotide at the opposite position on the primer when the primer is paired with the DNA strand. The primer is labeled at only one or at a few position(s), that are near or at the position opposite the test nucleotide. First, the primer and DNA strand are caused to pair. Then, conditions are applied to the primer-DNA pair that will cause retention of the label in the primer product in the presence of a match, but not in the presence of a mismatch. The test involves the use of a proofreading polymerase that removes the label on the primer if there is a mismatch when the primer and DNA are paired, but incorporates the label into a primer extension product if there is a match. In another aspect, the invention involves the use of an exonucleolytic agent that does not contain an associated polymerase activity, and monitors only the retention or excision of the label in the primer.

In another embodiment, the invention involves the use of an oligonucleotide primer that will create a base pair mismatch between a test nucleotide on the DNA strand and a nucleotide at the opposite position on the primer when the primer is paired with the DNA strand. The primer and DNA strand are caused to pair and, then, conditions are applied to the primer-DNA pair that will cause the mismatched nucleotide on the oligonucleotide primer to be cleaved and a labeled nucleotide or dideoxynucleotide complementary to the test nucleotide to be inserted.

The term “oligonucleotide” as used herein includes a molecule comprised of two or, preferably, more than three repeating units consisting of deoxyribonucleotides or ribonucleotides. The exact size of the molecule may vary according to its particular application and it may be synthetic or natural. The term oligonucleotide includes primers, extension products, tails and products complementary to primers, extension products and tails. Tails may also consist of synthetic, or non-naturally-occurring repeating units or nucleotides which may or may not be efficiently replicated by native DNA polymerases.

The term “primer” as used herein includes an oligonucleotide which, when paired with a strand of DNA, is capable of initiating the synthesis of an extension product in the presence of a suitable polymerization agent. Preferably, the primer is an oligoribonucleotide and most preferably is an oligodeoxyribonucleotide. The primer, however, may be other than a ribonucleotide. The primer must be sufficiently long to hybridize uniquely to the test region of the test DNA strand, and the primer must contain a labeled nucleotide at or near (1-3 nucleotides away from) the position opposite the test nucleotide of the test DNA strand. The exact length of the primer will depend on many factors, including the degree of specificity of pairing required, and the temperature and ionic strength during hybridization.

The term “proofreading polymerase” as used herein includes any moiety, e.g., an enzyme, that has the ability to that has the ability to catalyze the template-directed synthesis of DNA from deoxyribonucleotide triphosphates, and also to excise a mismatched nucleotide(s) at or near the 3′ terminus of a primer by means of an integral 3′ to 5′ exonuclease activity. The term includes enzymes such as DNA polymerase I from E. coli, phage Phi-80 DNA polymerase, and heat-stable polymerases from Bacerial and Archaeal species such as Pyrococcus furiosis (Pfu), Pyrococcus woesius (Pwo) Thermococcus litoralis (Tli, and Vent™ Polymerases) and Bacillus stearothermophius (Bst polymerase).

As used herein, the terms “DNA polymerase” and “polymerase” include any moiety, e.g., an enzyme, that has the ability to catalyze the template-directed synthesis of DNA from deoxyribonucleotide triphosphates. The term includes the above-mentioned proofreading polymerases, and also polymerases such as the Thermus aquaticus polymerase (Taq) that lacks 3′ to 5′ exonuclease activity.

The term “exonucleolytic agent” as used herein includes any moiety, e.g., an enzyme, that has the ability to differentially excise a matched and mismatched nucleotide(s) at the 3′ or 5′ terminus of a primer, when hybridized to a complementary strand of DNA. Suitable enzymes for this purpose may include proofreading DNA polymerases, Exonuclease III of E. coli, Lambda exonuclease, and related enzymes.

The term “terminal nucleotide” as used herein in referring to oligonucleotide primers refers to a terminal nucleotide at the either end of the primer. The term may be further qualified to specify the 3′ of 5′ terminal nucleotide. When the primer is hybridized to the test DNA, the nucleotide position opposite to the position of the test nucleotide on the DNA strand is located at or close to a terminal nucleotide.

The term “pairing” as used herein contemplates any and all methods of sequence specific pairing between the primer and a strand of DNA including the pairing of a primer with double stranded DNA, so long as an exonucleolytic agent may act on the product of such a pairing. Typically, however, a single stranded primer and a single strand of DNA will be paired by subjecting them to conditions which cause them to hybridize to one another. The primers are selected to be “substantially” complementary to the strands of each specific DNA sequence being tested. By substantially it is meant that the primer is sufficiently complementary to pair with the test DNA. The primer sequence then need not reflect the exact sequence of the test DNA. However, in a preferred embodiment, the primer is at least 16 nucleotides long and contains no mismatches with the complementary DNA strand except in certain instances at or close to the nucleotide position complementary to the test nucleotide.

The terms “match” and “mismatch” refer to the hybridization potential of paired nucleotides in complementary strands of DNA. Matched nucleotides hybridize efficiently, such as the classical A/T and G/C base pairs, and non-classical pairs such as iso-C/iso-G and Kappa/Xanthine. Mismatches are other combinations of nucleotides which do not hybridize efficiently.

The term “excised nucleotide(s)”, “excised nucleotide product(s)”, or “excision product(s)” used herein includes any nucleotide or combination of nucleotides with associated label (if applicable) that is removed from the terminal position of a primer by a proofreading polymerase or exonucleolytic agent. Examples include labeled deoxynucleoside 5′ monophosphates.

The term “extension product” used herein includes any derivative of a primer that includes additional nucleotides added by the action of a polymerase. Examples include n+1 single strand extensions of a primer, PCR products, RCA products, primer derivatives in which the 3′ terminus has been replaced by a dideoxynucleotide through excision and extension.

The term “label” as used herein includes any moiety capable of being detected, e.g., primary labels and secondary labels. Primary labels, such as radioisotopes (e.g., ³²P, ³³P, ³⁵S, or ¹⁴C), mass-tags, e-tags, and fluorescent moieties are signal generating reporter groups which can be detected without further modifications.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescence labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4 ′, 5′-Dichloro-2 ′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2 ′, 4 ′, 5 ′, 7′-Tetra-bromosulfone-fluorescein, Tetramethyl-rhodamine (TMR), Carboxytetramethylrhodamine (TAMRA), Texas Red, and Texas Red-X.

As used herein, the term “quencher” includes any moiety that is capable of absorbing the energy of an excited fluorescent label when in located in close proximity to the fluorescent label and capable of dissipating that energy without the emission of visible light . Examples of quenchers include, but are not limited to, DABCYL ( 4-(4′-dimethylaminophenylazo) benzoic acid) succinimidyl ester, diarylrhodamine carboxylic acid, succinimidyl ester (QSY-7), and 4 ′,5′-dinitrofluorescein carboxylic acid, succinirnidyl ester (QSY-33) (all available from Molecular Probes), quencherl (Q1; available from Epoch), or “Black hole quenchers” BHQ-1, BHQ-2, and BHQ-3 (available form BioSearch, Inc.).

The term “mass-tag” as used herein refers to any moiety that is capable of being uniquely detected by virtue of its mass, for example, using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, 5,650,270, the contents of each of which are incorporated herein by reference. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “electrophoretic-tag”, or “e-tag” as used herein, include any moiety that is capable of being uniquely detected by virtue of its charge-to-mass ratio using electrophoretic separation techniques. Such electrophoretic separation techniques include capillary electrophoresis and separation in polymer- or gel-filled microchannels in manufactured “chips” or devices made of silica, glass, plastic, or other materials (e.g., “sequencing chips”). Examples of e-tags include charged molecules of the type described in PCT application WO066607A1, and may be attached to DNA primers by means of the labeling methods described herein.

The term “substrate”, as used herein refers to any material or macromolecular complex to which a molecule complementary to a tail can be attached, and which can be separated from an aqueous solution by virtue of its solidity or insolubility under an appropriate condition. Examples of commonly used substrates include, but are not limited to, glass surfaces, silica surfaces, plastic surfaces, metal surfaces, surfaces containing a metalic or chemical coating, membranes (eg., nylon, polysulfone, silica), micro-beads (e.g., latex, polystyrene, or other polymer), porous polymer matrices (e.g., polyacrylamide gel, polysaccharide, polymethacrylate), macromolecular complexes (e.g., protein, polysaccharide).

Preparation of the Oligonucleotide Primers/Tails

Precursors of the labeled oligonucleotide primers (including tails) of the invention or the oligonucleotide primers themselves may be prepared using any suitable method, such as, for example, methods using phosphotriesters and phosphodiesters well known to those skilled in the art. In one automated embodiment, diethylphosphoramidites are used as starting materials and may be used for synthesis of oligonucleotides as described by Beaucage and Caruthers, 1981, Tetrahedron Letters, 22:1859-1862. One method for synthesizing oligonucleotides on a modified solid support is described in U.S. Pat. Nos. 4,458,066 and 4,500,707, the contents of which are incorporated herein by reference. It is also possible to use a precursor primer or a primer which has been isolated from a biological source (such as a restriction endonuclease digest of plasmid or phage DNA).

Labels may be attached to primers by any suitable chemical or enzymatic method. For example, N-hydroxysuccinimide esters of fluorescent labels can be conjugated to linkers containing a primary amine (e.g., ‘N-hydroxysuccinimide ester labeling 5′-aminoalkyl DNA oligomers: reaction conditions and purification' J. Chromatogr. 806, 93-95, 1998). For this purpose, linkers containing a primary amine can be attached at the 5-position on pyrimidine bases and at a 7-deaza- or 8-position of purine bases. Compounds such as 5′-Dimethoxytrityl-N-dimethylformamidine-5[N(trifluoroacetylaminohexyl)-3-acrylimido]-2′-deoxyCytidine, 3′-succinoyl-long chain alkylamino-CPG 1000 are useful for constructing oligonucleotides with an amino-linker at the 3′ terminus. DNA polymerases such as the Klenow fragment of DNA polymerase I may be used to add certain labeled nucleotides to the 3′ end of a precursor primer in the presence of the suitable template DNA strand. Alternatively, labels may be incorporated directly into the chemical monomers from which primers are synthesized, which eliminates the necessity for post-synthetic labeling. For example, 5′-Dimethoxytrityloxy-5-[N- ((3′,6′-dipivaloylfluoresceinyl)-aminohexyl)-3-acryimido]-2′-deoxyUridine-3′-succinoyl-long chain alkylamino-CPG 500 can be used as a starting material to produce oligonucleotides with fluorescein attached to a 3′ terminal thymidine residue.

Tails composed of repeating units other than deoxyribonucleic acid can be synthesized using the appropriate chemistry and monomeric precursors. For example, PNA derivatives can be synthesized using tBoc and Fmoc methods.

Detection Methods

Detection methods well known to those skilled in the art may be employed to determine the presence or absence of label in the extension product. For example, the reaction products may be fractionated by methods such as gel electrophoresis, dialysis, gel filtration, ion exchange chromatography, solvent extraction and differential precipitation to separate excised nucleotides, primers, and extension products. These components (or mixtures thereof) can then be independently tested for the presence or absence of a label. For example, extension products may be separated from the smaller primers and nucleotide excision products on QIAquick silica membranes (using a standard protocol available from the manufacturer, QIAGEN Inc., Valencia, CA). In this procedure, the smaller molecules are removed during the washing procedure, and the purified extension products are eluted in a low salt buffer, or water. Extension products may captured on a substrate (e.g., a silica, plastic or metal surface, membrane, micro-bead, or porous polymer matrix) by hybridization using oligonucleotides complementary to the extension products or to tail sequences. The substrate may be treated with the products of the test reaction under conditions that would allow the extension products to hybridize to the complementary DNA on the substrate. The substrate then would be tested for the presence or absence of any label attached via hybridization to the complementary strand.

The presence or absence of a label in excised nucleotides and extension products may also be detected by direct fluorescence or by using fluorescence polarization. When a fluorescent sample is exposed to polarized light at its absorption wavelength, fluorophores of appropriate transition moment orientation are excited. The fluorescent light emitted from such molecules is polarized like the incident light, but the polarization decreases by the extent to which the molecules have rotated during the time between absorbing and emitting light. Consequently, the decrease in polarization measures the rotation of the molecules during the lifetime of the excited state. In the situation where a fluorescent label is retained during the synthesis of the extension product, the extension product containing the label will undergo a slower rotational Brownian motion because of its higher effective volume/mass and an increase in the fluorescence polarization will be observed. In the situation where the fluorescent label is excised prior to the synthesis of the extension product, the free fluorescent label will undergo a faster rotational Brownian motion because of its lower effective volume/mass and a decrease in the fluorescence polarization will be observed. The fluorescence polarization measurements may be performed using any suitable instruments available in the art including the FluoroMax-2 instrument (available from Instruments S.A., Edison, N.J.), FP777 spectrofluorimeter equipped with a microcomputer-assisted polarization measurement module and a Peltier temperature regulation system (available from Jasco, Tokyo), and the Analyst HT microplate reader (available from Molecular Devices Corp.). Most of these instruments may also be used to make direct fluorescence measurements of a sample in a microtiter plate (e.g., plastic plates with 96, 384, 1536 wells). Numerous instrumentation options are available to permit direct fluorescence measurements to be obtained from samples on microbeads (e.g., fluorescence activated flow sortering, and etched fiber optic arrays), microarrays (e.g., confocal microscopy and CCD imaging), gels, capillary electrophoresis and microfluidic devices (e.g., optical systems using laser excitation and photomultiplier or CCD detection), and large format membranes (optical scanning and CCD detection systems).

The presence or absence of a label in excised nucleotides and extension product may further be detected using mass spectrometry to produce a direct mass measurement (as opposed to gel electrophoresis that separates ions according to their mobilities correlating with their masses and charges). Mass spectra can be acquired in a very short period of time, e.g., in minutes, seconds, or fractions of a second. Different mass spectrometry methods can be used depending on the chemical nature of the molecules being analyzed. For example, short extension products, oligonucleotide and oligopeptide mass-tags can be efficiently detected using matrix-assisted laser desorption/ionization time-of-flight mass spectrometry (MALDI-TOFMS) (as described in, for example, Fei, Z. et al. (1998) Nucleic Acids Res. 26:2827-2828). Typical MS detection techniques for electrophore mass-tags include gas chromatography electron-capture mass spectrometry (GC-EC-MS), laser induced electron capture time of flight mass spectrometry (LI-EC-TOF-MS), and laser desorption electron capture time of flight mass spectrometry (LD-EC-TOF-MS) (Giese, R. W., ‘Electron-capture mass spectrometry: recent advances’ J. Chromatogr., 892, 329-346, 2000). The latter two methods are particularly useful for the detection of mass tag labeled products deposited or captured on metal or plastic surfaces (Xu, et al., ‘Electrophore Mass Tag Dideoxy DNA Sequencing’, Analytical Chemistry, 69, 3595-3602, 1997; Wang, P. & Giese, R.W., ‘Laser-Induced Electron Capture Mass Spectrometry’ Analytical Chemistry 72, 772-776, 2000).

The presence or absence of a label in excised nucleotides and extension products may also be detected using a dialysis system such as the flat plate dialysis system described in U.S. Provisional Patent Application Serial No. 60/228,239, filed on Aug. 25, 2000, the contents of which are incorporated herein by reference. Briefly, the products of the PCR extension reaction may be injected, e.g., from a needle, through a syringe docking port into a dialysis chamber and into contact with a first side of a dialysis membrane. A dialysis solution may then be applied to a second side of the dialysis membrane opposite the first side of the dialysis membrane. The molecular weight cut-off for the dialysis membrane should be selected such that passage of the PCR extension product and the primer through the membrane does not occur (or occurs only slowly), while at the same time passage of the mismatched and, thus, cleaved labeled nucleotide is allowed (or occurs rapidly). The dialysate may then be collected and the presence of the label in the dialysate may be determined using any of the assays described herein, e.g., direct fluorescence measurement, or mass spectrometry.

In another embodiment, the cleaved labeled nucleotide can be separated by diffusion and subsequently detected using a microfluidic system that does not require a semipermeable membrane. This can be accomplished by the merging and subsequent separation or two liquid channels (one containing the sample and the other containing water, and organic solvent, or some other suitable “diffusate”) under conditions that avoid convective mixing, but which permit the size-dependent diffusion of the molecular components in the sample to occur while the two liquid streams are in contact. Another approach involves the interposition of a nano-fabricated screen between two liquid channels in a microfabricated device. In such devices, the excised nucleotide will preferentially migrate into the diffusate (relative to the extension product), and this can be monitored as before using a suitable detection method, e.g., direct fluorescence measurement, or mass spectrometry.

Excised nucleotides containing fluorescent or mass-tag labels are typically more hydrophobic than the starting primers or other molecules in the reaction mixture (because of the attached hydrophobic label) and, thus, may be isolated and detected based on this physical property. For example, the products of the PCR extension reaction may be exposed to a an organic solvent, e.g., an allyl alcohol, organic acid, ether, ester, or oil. The hydrophobic labeled nucleotide will diffuse (partition) into the hydrophobic solution while the PCR extension product and the primer will not, thus, effecting the separation of the labeled nucleotide. The presence of the label in the organic phase may be determined using any of the assays described herein, e.g., direct fluorescence, fluorescence polarization, or mass spectrometry.

Exo-Proofreading SNP Assay

According to the preferred embodiment of this invention, a proofreading polymerase is used to determine whether a primer contains a nucleotide that is complementary to, or not complementary to, the test nucleotide in the DNA strand. Typically, a single stranded primer when hybridized to a longer single strand of DNA in the presence of a polymerase and nucleoside triphosphates (at an appropriate temperature and pH, and in the presence of suitable ions) will allow the synthesis of an oligonucleotide attached to and extending from the primer (an extension product), the oligonucleotide being complementary with the single strand of DNA. Most polymerases will not efficiently catalyze extension of a primer from a mismatched terminal base pair. Rather, if it contains an integral 3′ to 5′ exonuclease activity, the polymerase will excise the mismatched base pair and then initiate the synthesis of an extension product from a penultimate matched pair. This process is commonly referred to as “proofreading”, or “editing” and is highly accurate. The error rate of proofreading polymerases (extension of a mismatched nucleotide) is typically on the order of 1 in 10⁻⁵ or less (Kunkel, et al., J. Biol. Chem. 256: 1539-1545; Mattila et al., Nucl. Acids Res. 19:4967-4973; Cline, et al., Nucleic Acids Res. 24: 3546-3551). Mutant forms of proofreading polymerases with even lower error rates (such as the “anitmutator” form of T4 polymerase), have been described in the literature (Drake, et al., Nature 221:1128).

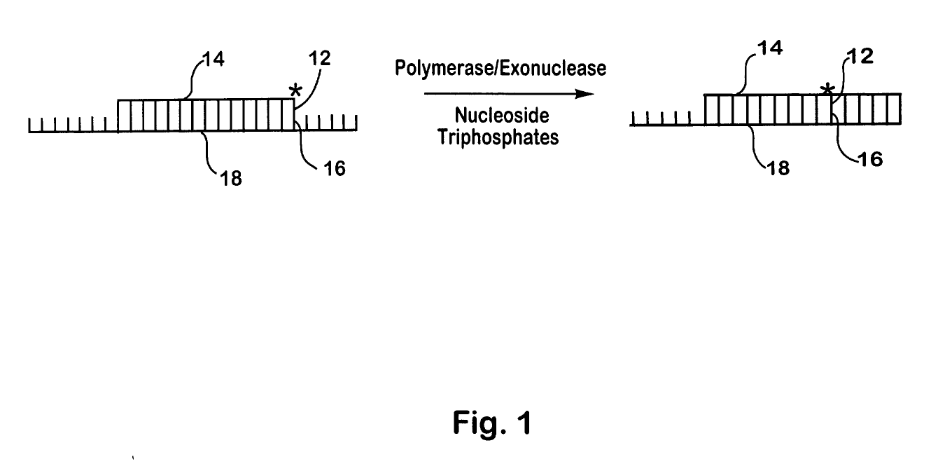

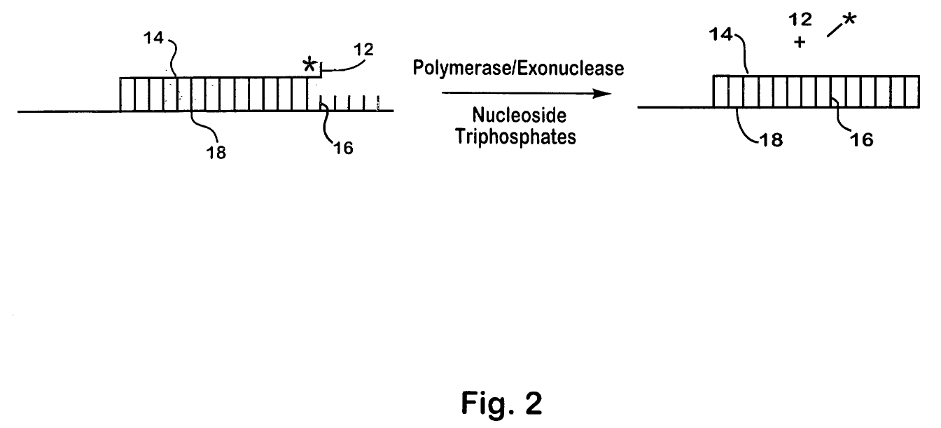

As shown in FIG. 1, if there is complementary base pairing (a match) between the labeled terminal nucleotide 12 of the primer 14 and the test nucleotide 16 on the test DNA 18, an extension product will be synthesized and the labeled nucleotide (*) will be retained. However, as shown in FIG. 2, if there is a mismatch between the labeled terminal nucleotide 12 of the primer 14 and the test nucleotide 16, then an extension product will be synthesized but only after excision of the mismatched labeled nucleotide opposite the test position in the DNA strand.

The synthesis of the extension product may be according to methods well-known to those skilled in the art. For example, if a deoxyribonucleotide extension product is being synthesized, the hybridized primer-DNA strand must be treated with a proofreading polymerase in the presence of deoxyribonucleoside triphosphates (dATP, dCTP, dGTP, dTTP).

According to a preferred embodiment, the primer carries a labeled nucleotide at or close to its 3′ end. Typical labels include mass-tags, biotin, or fluorescent moieties. The labeled nucleotide is at the position opposite the test nucleotide on the test DNA strand when the primer is hybridized to the test DNA strand.

By using a proofreading polymerase, which excises a mismatched nucleotide in the primer before initiating the synthesis of an extension product, and by subjecting the hybridized primer-DNA to conditions that permit excision and extension, the presence or absence of a specific nucleotide on a strand of DNA may be determined with high accuracy. For example, assume that a normal gene includes the following known sequence: 5′TTAAGATCGAATTGGCTCACGTT3′(SEQ ID NO:1). Also assume a disease state is due to or correlated with a substitution at a single test nucleotide position, underlined: 5′TTAAGATCGAATTGGCCCACGTT3′(SEQ ID NO:2). A primer that could be used to detect the presence or absence of the disease state then would be: 5′GATCGAATTGGCC*3′(SEQ ID NO:3), in which the terminal “C” is labeled (the * denotes the label). This primer is capable of hybridizing with the complementary strand corresponding to either of the foregoing DNA sequences. However, when the primer hybridizes to the DNA sequence characteristic of the normal state, there will be a mismatch at the terminal end of the primer, an C being paired with an A (on the complement of SEQ ID NO:1). If that hybridized primer-DNA strand is treated with a proofreading polymerase then an extension product will be formed, but only after excision of the labeled “C” residue of the primer. On the other hand, when the primer is hybridized with the DNA sequence characteristic of the disease state, there is a match between the terminal nucleotide of the primer and the test nucleotide on the DNA strand (C-G). That hybridized primer-DNA strand will initiate the synthesis of an extension product with retention of the labeled “C” residue in the presence of the proofreading polymerase of the invention.

To determine whether a sample of test DNA carries the DNA characteristic of the healthy state or the disease state, the labeled primer is added to a sample of test DNA under conditions allowing the primer to hybridize to the test DNA. A proofreading polymerase and nucleoside triphosphates then are added and the mixture is subjected to conditions that allow excision and synthesis of an extension product. It is then determined whether label is present or absent in the resulting extension product (and/or excision product). If label was retained in the extension product, then there was a match indicating the presence of the DNA characteristic of the disease state. If label was lost in the extension product, then there was not a match and the nonexistence of the DNA characteristic of the disease state is established.

To improve the accuracy of the test, it is desirable to use two labeled primers simultaneously so that a positive signal is obtained from both alleles at the test nucleotide. In the present example, the additional primer would have the sequence: 5′GATCGAATTGGCT^{#}3′(SEQ ID NO:4) in which the terminal “T” is labeled using a label that is distinguishable from the label on SEQ ID NO:3 (the # denotes the different label). In this case, application of the test using both primers (SEQ ID NO:3 & 4) and a sample from an individual who is homozygous for the normal allele would produce an extension product containing the # label and an excised nucleotide containing the * label; application of the test using a sample from an individual who is homozygous for the disease allele would produce an extension product containing the * label and an excised nucleotide containing the # label; application of the test using a sample from an individual who is heterozygous for both alleles would produce two labeled extension products (one containing the # label and one containing the * label), and two excised nucleotide products (one containing the # label and one containing the * label). If required, one or two additional labeled primers with distinguishable labels could be used to discriminate the one or two additional possible variants (A and G) at the test position.

In practice, it is preferable to amplify the amount of extension and excision product generated in the reaction by, for example, the use of PCR (as described in U.S. Pat. No. 4,683,195, the disclosure of which is incorporated herein by reference). Such PCR amplification allows a detectable signal to be generated in a rapid manner in a single reaction using easily obtained samples such as tissue swabs, blood cells, or crude genomic DNA preparations. The PCR would be accomplished using a proofreading polymerase instead of the typical Taq polymerase which lacks 3′ to 5′ exonucleolytic activity. Also, such a PCR amplification would be accomplished using as a first primer, the labeled primer of the invention, and as a second primer (reverse primer), that is complementary to a region “downstream” from the first primer. Preferably these primers are at lease 16 nucleotides long and most preferably are 20-25 nucleotides long. This length will insure specific hybridization at the desired locations in genomic DNA.

The extension and excision products may also be amplified in an isothermal reaction in which new single-stand copies of the DNA molecules in the sample are faithfully generated, such as occurs in a rolling circle amplification (RCA) reaction. In the case of RCA, the strand of DNA of the sample being analyzed must be a circular molecule, or it must be circularized through some artificial means, eg., by ligation of a linear fragment having blunt or cohesive ends derived from the sample (a restriction endonuclease derived fragment, or a hydrodymanically or enzymatically sheared fragment, or a PCR product), or by ligation of a “padlock probe” that is capable of hybridizing with the DNA molecule of the sample.

Single Nucleotide Replacement Assay

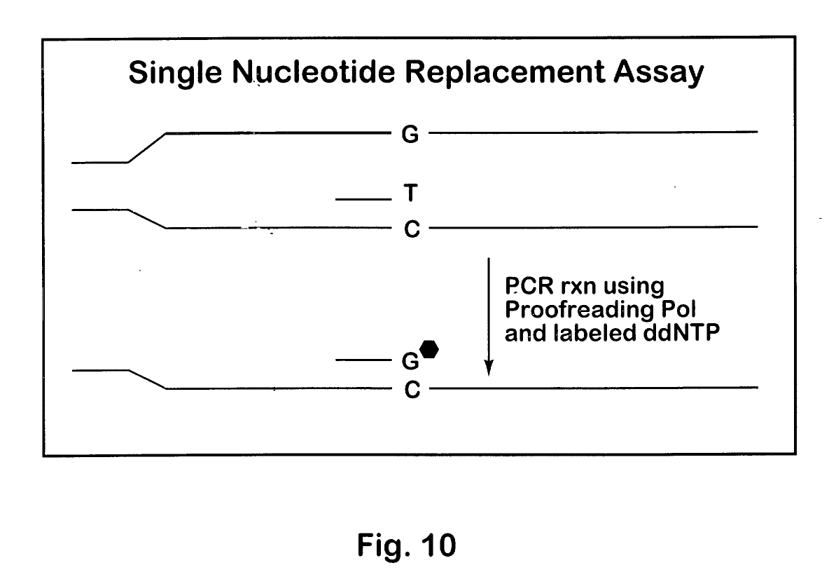

In another embodiment, the present invention features a method which involves using an oligonucleotide primer that contains a nucleotide that hybridizes opposite the test nucleotide on the DNA strand, but which nucleotide is not complementary to the test nucleotide on the DNA strand. As shown in FIG. 10, when the primer and DNA strand are caused to pair, conditions may then be applied to the primer-DNA pair that will cause the mismatched nucleotide on the oligonucleotide primer to be cleaved and a labeled nucleotide or dideoxynucleotide complementary to the test nucleotide to be inserted, replacing the excised nucleotide.

For example, the primer-DNA pair may be incubated in the presence of a proofreading polymerase and labeled nucleotides or labeled dideoxynucleotides. In the situation when labeled dideoxynucleotides [e.g., dideoxy adenosine triphosphate (ddATP), dideoxy cytosine triphosphate (ddCTP), dideoxy guanosine triphosphate (ddGTP), or dideoxy thymidine triphosphate (ddTTP)] are used, the incubation may be performed in the absence of nucleotides and each of the four dideoxynucleotides may be labeled with a different label. The identity of the label will be indicative of the identity of the dideoxynucleotide that is inserted. Since the dideoxynucleotide is complementary to the test nucleotide on the DNA strand, the identity of the test nucleotide may also be determined.

Quencher-Fluorophore Assay

In another aspect, the invention features a method which involves using an oligonucleotide primer that contains a quencher moiety adjacent to, e.g., immediately adjacent to or near, the position of a fluorophore-labeled nucleotide that is opposite to the test nucleotide when the primer and the strand of DNA are paired. For example, the quencher moiety may be at least 1, 2, 3, 4, 5, 6, 7, 8, 9, or 10 nucleotides away from the position containing the fluorophore label, which is opposite to the test nucleotide when the primer and the strand of DNA are paired. Because of the close juxtaposition of the quencher and the fluorophore in the primer (well within the Forster radius) the excitation energy of the fluorophore will be effectively transferred to the quencher by non-radiative FRET, but since the quencher emits this energy as heat instead of light, the primer will be non-fluorescent. The DNA sample is incubated with this quencher/fluorophore containing primer in the presence of a proofreading polymerase and nucleoside triphosphates, and the mixture is subjected to conditions that allow excision and synthesis of an extension product. The fluorescence of the sample is then monitored.

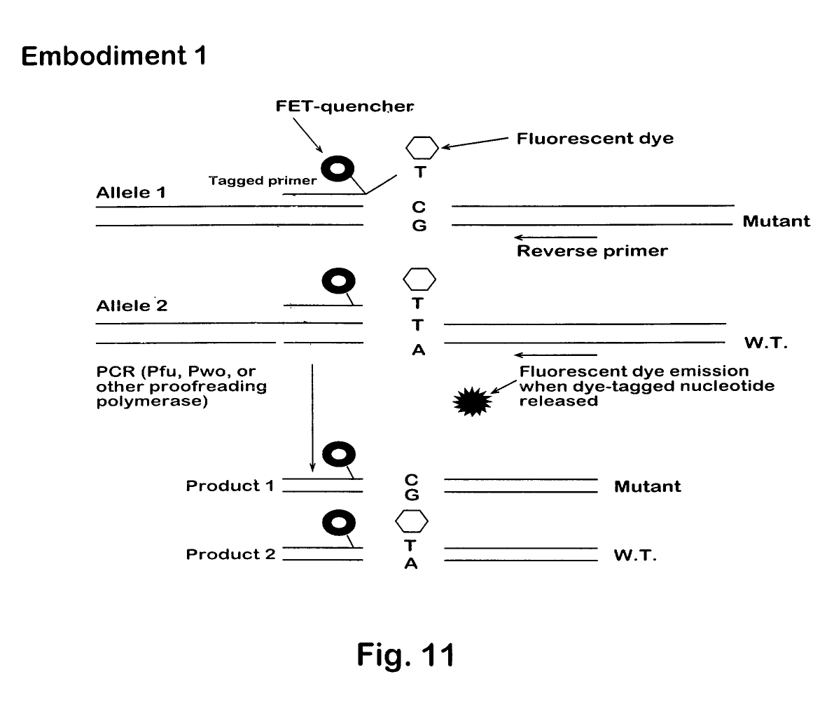

As shown in FIG. 11, in the case of the wild type allele 2, the nucleotide labeled with the fluorescent dye will be incorporated into the extension product 2, thus maintaining the juxtaposition of the quencher moiety and the fluorescent dye in close proximity to each other (e.g., 1, 2, 3, 4, 5, 6, 7, 8, 9, or 10 nucleotides apart) causing the quenching to be maintained, so that the sample will be non-fluorescent. In the case of the mutant allele 1, the nucleotide labeled with a fluorescent dye will be excised, and therefore will not be incorporated into the extension product 1. In this case, the fluorescent dye will be liberated into solution, and because it will no longer be constrained to a position within the Forster radius required for FRET, the fluorescence will no longer be quenched, and the sample will fluoresce.

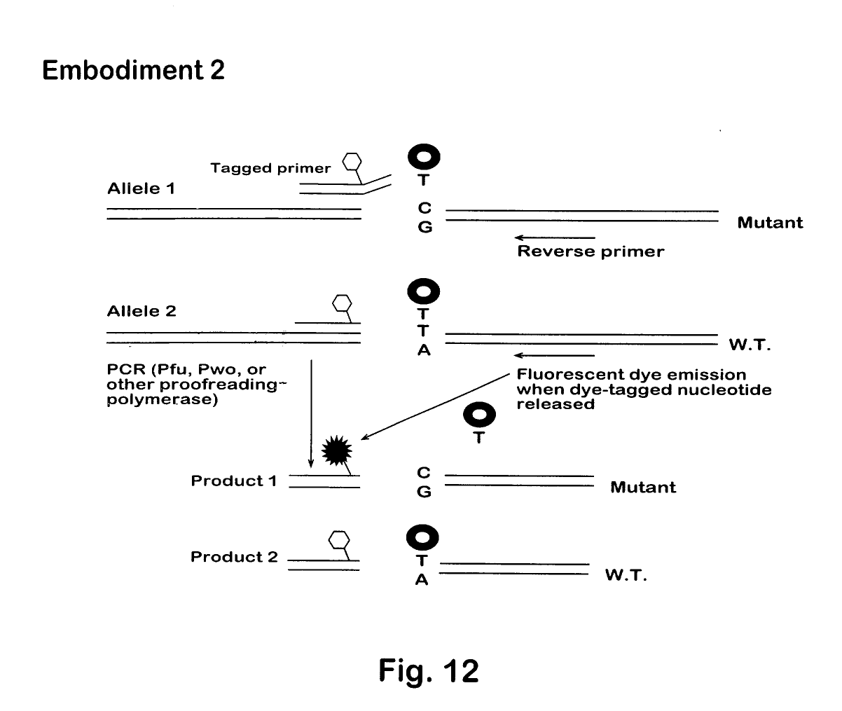

In another embodiment, schematically represented in FIG. 12, the opposite configuration may be used. Namely, the oligonucleotide primer may contain a fluorophore (e.g., a fluorescent label/dye) adjacent to (e.g., immediately adjacent to or within 1, 2, 3, 4, 5, 6, 7, 8, 9, or 10 nucleotides away from) the position of a quencher-containing nucleotide that is opposite to the test nucleotide when the primer and the strand of DNA are paired. Because of the close juxtaposition of the fluorophore and the quencher in the primer the excitation energy of the fluorophore will be effectively transferred to the quencher by non-radiative FRET, and the primer will be non-fluorescent. The DNA sample is incubated with this quencher/fluorophore containing primer in the presence of a proofreading polymerase and nucleoside triphosphates, and the mixture is subjected to conditions that allow excision and synthesis of an extension product. The fluorescence of the sample is then monitored.

As shown in FIG. 12, in the case of the wild type allele 2, the nucleotide labeled with a quencher moiety will be incorporated into the extension product 2, thus maintaining the juxtaposition of the quencher moiety and the fluorescent dye in close proximity to each other and causing the quenching to be maintained, so that the sample will be non-fluorescent. In the case of the mutant allele 1, the nucleotide labeled with the quencher will be excised, and therefore will not be incorporated into the extension product 1. In this case, the quencher will be liberated into solution, and because the fluorescent dye will no longer be constrained to a position within the Förster radius required for FRET, the fluorescence will no longer be quenched, and the sample will fluoresce. Any fluorescent dye and quencher combination may be used for this application, as long as the quencher is capable of effectively absorbing the electromagnetic energy emitted by the fluorophore by FRET when attached at a minimum separation of one nucleotide from the fluorophore.

The quencher and fluorescent label moieties may be incorporated into the primer using art known techniques as described previously (e.g., by conjugation of an N-succinimide ester with a primary amine, or by incorporation into CPG precursors or phosphoramidites). The quencher moiety and the fluorescent dye may be attached to the primer using linker molecules, the length and points of attachment of the linkers being of such a nature that they do not affect the ability of the bases to form hydrogen bonds during base pairing, and also of such a nature that they do not impair the functioning of the proofreading and polymerization activities of the polymerase. For example, linkers with a length of approximately 9 carbon atoms may be attached to the 5-position of a pyrimidine base, or to the 8-position, or a 7-deaza position, of a purine base.

In another embodiment, the presence or absence of the mismatch may be determined by using non-radiative fluorescent resonance energy transfer (FRET, see Cardullo et al. (1988) Proc. Natl. Acad. Sci. USA 85:8790-8794) between two suitable fluorescent labels. Using this technique, the fluorescent labels would allow effective FRET only if both fluorescent labels are maintained in close proximity. The oligonucleotide primer will contain two fluorophores adjacent to each other (e.g., immediately adjacent, or within 1, 2, 3, 4, 5, 6, 7, 8, 9, or 10 nucleotides apart). The first fluorescent label (absorbing light at a first wavelength and normally emitting light at a second wavelength) is attached to the oligonucleotide primer opposite to the test nucleotide when the primer and the DNA sample are paired. Because of the close juxtaposition of the second fluorophore (which absorbs light at the second wavelength and emits light at a third wavelength) the excitation energy of the first fluorophore will be effectively transferred to the second by non-radiative FRET. The DNA sample is incubated with this double fluorophore containing primer in the presence of a proofreading polymerase and nucleoside triphosphates, and the mixture is subjected to conditions that allow excision and synthesis of an extension product. The fluorescence of the sample is then monitored.

If there is base-pair match at the test nucleotide position, both fluorescent labels will be incorporated into the extension product, thus maintaining their close proximity to each other and illumination of the sample at the first wavelength will result in fluorescence emission to at the third wavelength (and not the second) due to the occurrence of non-radiative FRET. If the nucleotide labeled with the first fluorophore is mismatched, it will be excised, and illumination of the sample at the first wavelength will result in fluorescence emission to at the second wavelength, due to the absence of a FRET-induced shift.

Tails

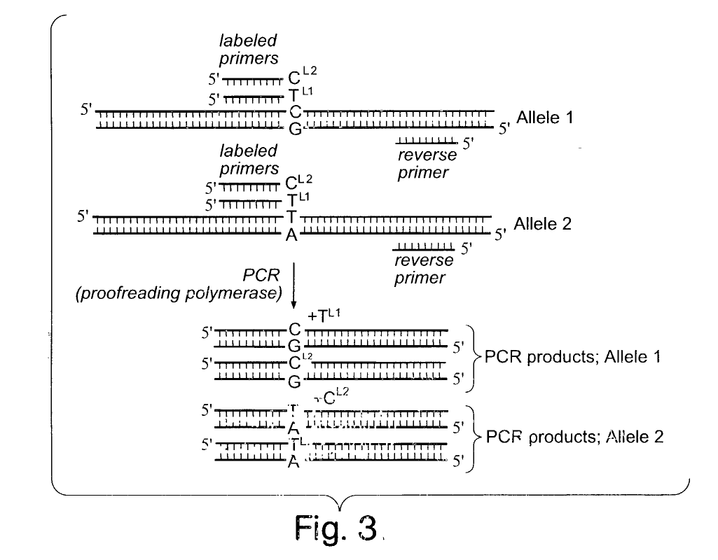

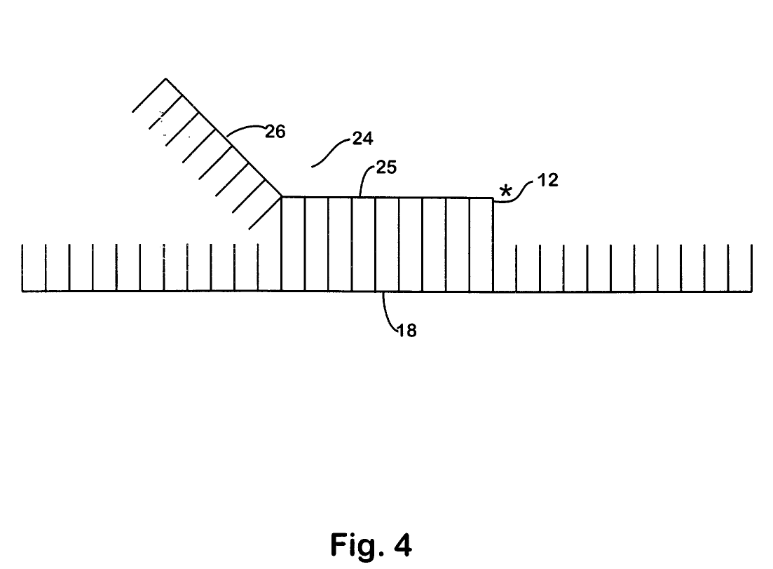

According to another embodiment of the invention, the test may be implemented by using primers with tails. For example, as shown schematically in FIG. 3, a primer 24 having primer portion 25 and a tail portion 26 attached to and extending from the end of the primer portion 25 opposite the labeled terminal nucleotide 12 is used. Preferably, the tail portion 26 is unique and is non-complementary with the test DNA. FIG. 3 shows such a primer hybridized to a longer strand of DNA 18. When using the labeled primer 24 of the invention and a proofreading polymerase, an extension product 27, having three portions, is formed (FIG. 4). The extension product will include the extension portion 28, the primer portion 25, labeled or unlabeled depending on the test nucleotide, and the tail portion 26.

Improvements to the speed and sensitivity of the assay may be achieved using such primers having tails. The presence or absence of label in the primer portion of the extension product may be detected by using a substrate 30 containing a great excess of oligonucleotide complementary to the tail portion 26. Because such complementary oligonucleotide DNA 32 may be synthesized inexpensively in great quantity and therefore may be applied to the substrate in great excess (FIG. 4), the rate and amount of hybridization between the tail portion 26 of the extension product 27 and the complementary oligonucleotide 32 on the substrate is enhanced.

Most preferably, the oligonucleotide of the tail and the oligonucleotide complementary to the tail both consist of repeating units of complementation. Most preferably, the tail portion 26 is a polymer consisting of repeating units of an oligonucleotide 14 nucleotides long, and the complementary oligonucleotide 32 is a polymer consisting of repeating units of an oligonucleotide that is also 14 nucleotides long. The use of such repeating units of complementation favorably affects the kinetics of hybridization, further increasing the speed and the sensitivity of the assay. However, the repeating units of complementarity may be composed of molecules other than nucleotides (e.g., PNA).

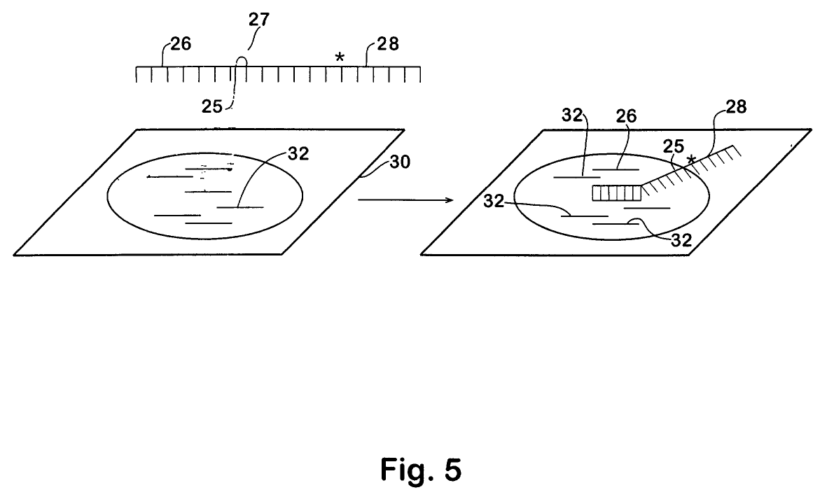

A substrate having attached to it a plurality of polymers 33 of such repeating units 34 of complementation is shown schematically in FIG. 5. Preferably, the plurality of polymers 33 are covalently linked to the substrate at a very high concentration to form a solid solution that presents a great many available hybridization sites, unobstructed by the substrate to which the polymers are attached. These substrates with attached polymers may be dried out and stored for considerable periods.

The products and methods of the invention may be used advantageously to determine allelic variation in genotyping studies. For example, if allelic variation is due to a single nucleotide substitution (or is correlated with such a substitution), then test DNA can be treated using primers for both alleles to determine whether an individual is homozygous or heterozygous with respect to those alleles. Such a test is performed advantageously using primers for each allele having tails differing from one another so that only a single test carried out in a single vessel is necessary.

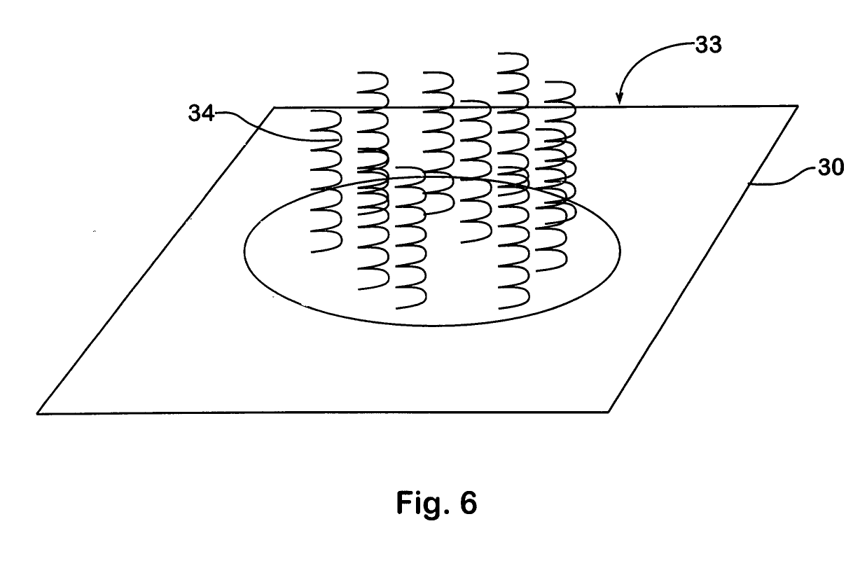

To accomplish this, two or more primers are constructed as shown in FIG. 6. Each primer has a primer portion P that is complementary to the same DNA, except that the labeled terminal nucleotide on each of the primers is different. The labeled terminal nucleotide on one of the primers is complementary to the nucleotide determining one allele and the labeled terminal nucleotide on the other primer is complementary to the nucleotide determining the second allele. In the example shown, the labeled terminal nucleotides are cytosine and adenosine (C and A, respectively).

At the opposite end of each of the primers is attached a unique tail. By “unique” it is meant that a sequence complementary to one tail will not hybridize with the other tail. Moreover, neither of the tails and neither sequence complementary to the tails should be capable of hybridizing with the test DNA. It is believed that a single nucleotide substitution on an oligonucleotide 14 nucleotides long is sufficient to prevent cross hybridization. Preferably there are at least two nucleotide substitutions to distinguish each tail. As is understood by those skilled in the art, the synthesis of a set of thousands of such unique tails greater than six nucleotides long is possible.

The designations for the primers shown in FIG. 6 are T₁, P₁ C* and T₂ P1 A* the T signifying tail and the subdesignation signifying the sequence of the tail; the P signifying primer portion and the subdesignation signifying the sequence of the primer; and the last letter signifying the labeled terminal nucleotide. Thus, T₁, P1 C* stands for tail sequence number 1, primer sequence number 1, and a cytosine terminal nucleotide. T₃ P₂ A* would stand for tail sequence number 3, primer sequence number 2 and adenosine as a terminal nucleotide.

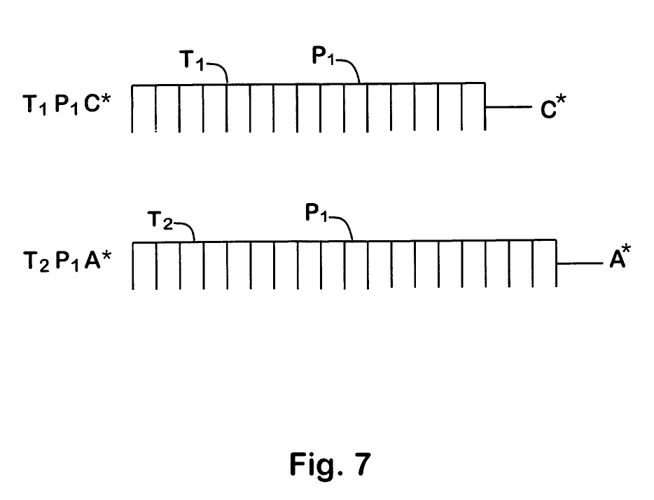

The primers shown in FIG. 6 (T₁ P₁ C* and T₂ P1 A*) are added to test DNA under conditions that allow the primers to hybridize with the test DNA. Then, the hybridized primer-DNA may be treated with a proofreading polymerase and nucleoside triphosphates under conditions that allow the synthesis of an extension product with retention of label if there is a match at the labeled terminal nucleotide. Thus, if the test DNA has a G at the test nucleotide, which is complementary to the labeled terminal nucleotide of the primer T₁, P1 C*, then there is a match and an extension product will be synthesized with retention of the label. Likewise, if the test DNA has a T at the test nucleotide which is complementary to the terminal nucleotide of the primer T₂ P1 A*, then there is a match and an extension product will be synthesized, with retention of the label. The sample containing the extension products then is applied to a substrate having at different locations an oligonucleotide complementary to tail number 1 (T₁) ′ and an oligonucleotide complementary to tail number 2 (T₂ ′) (FIG. 7). In some cases, e.g., with microbead substrates, it maybe advantageous to attach the different tail complements to separate substrates (separate beads) to facilitate analysis in flow based fluorescence detection devices, or by adsorption on etched fiber optic bundles. The extension product will hybridize at T₁′ via hybridization of tail number 1 to the T₁′ oligonucleotide and extension product also will hybridize to spot T₂ ′ via hybridization of tail number 2 to the oligonucleotide at T₂ ′. The presence of label at both locations would indicate a heterozygous individual. If, on the other hand, label is detected only at spot T₁ ′, then the individual carries only a G at the test nucleotide. Likewise, if label is only detected at spot T₂ ′,then the individual carries only a T at the test nucleotide position. Thus, the genotype of an individual at a single locus may be determined in a single test, two alleles being tested for simultaneously.

It will be understood by those skilled in the art that the genotype could have been tested by using primers having the same tail, rather than unique tails. To accomplish this, the primers must be tested separately with separate samples of test DNA. It, however, is an advantage of the invention that by using unique tails, any number of alleles or loci may be tested for simultaneously. Thus, tests for different genes and tests for multiple alleles on different genes may be accomplished simultaneously according to the invention. For example, a plurality of primers may be constructed, including primers complementary to different genes.

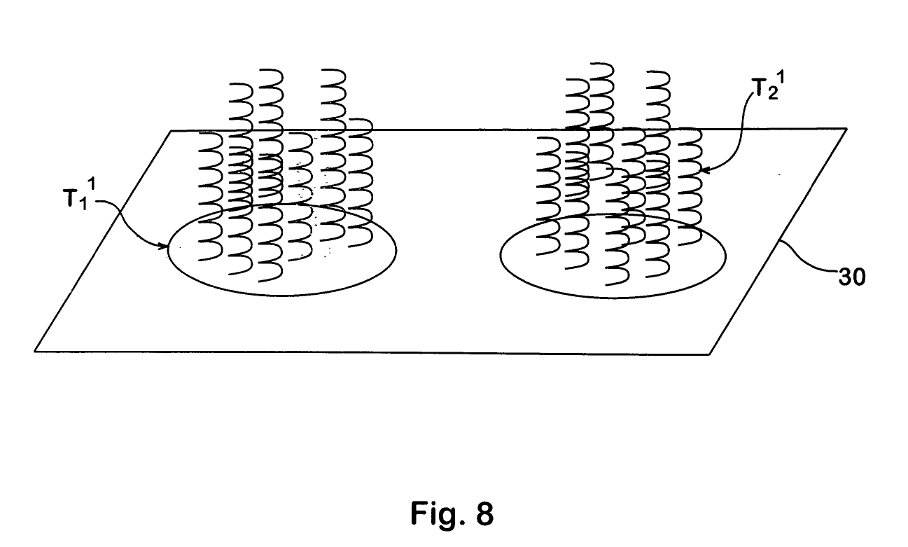

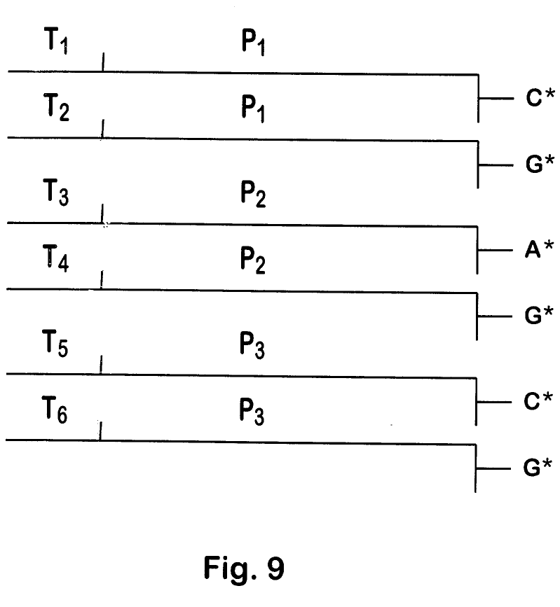

FIG. 8 depicts a set of primers for three genes, each gene having two alleles. T₁ P₁ C* and T₂ P₁ G* are complementary to the same gene, but to different alleles; T₃ P₂ A* and T₄ P₂ G* are complementary to the same second gene, but to different alleles; and T₅ P₃C* and T₆ P₃ G* are complementary to a third gene, but also to different alleles of that gene. Each of the primers has a unique tail (T₁, T₂, T₃, T₄, T₅, and T₆), and the terminal nucleotide of each primer is labeled. When this set of primers is mixed with a single sample of test DNA, only those primers that have hybridized to the test DNA and have matching nucleotides at the terminal end of the primer are capable of initiating the synthesis of an extension product retaining the labeled nucleotide. After the conditions for the proofreading polymerase reaction have been applied, the label on any unreacted primers may be removed by use of a potent exo- or endonuclease which prefers single-stranded DNA, such as mung bean nuclease or Exonuclease I. Alternatively, label on any unreacted primers may be removed by separating extension products from unreacted primers. Next, the products of the reaction may be placed in contact with specific oligonucleotides, complementary to the unique tails, spotted at different locations on a substrate. Then, the existence of label on the reacted primers is determined by looking for the presence of label on the substrate, potentially present due to reacted primers hybridizing via their tails to the substrate. The existence of label at a particular location on the substrate indicates that label was retained on the primer portion of an extension product, the primer being identified by its unique tail complementary only with the oligonucleotide at the particular location. Thus, the presence or absence of each of the various genes and multiple alleles may be tested simultaneously using a single sample of test DNA.

For the implementation of these first two embodiments, the complementary DNA attached to the substrate may be complementary to at least one of the following: a portion of the primer (including complementation to only the tail portion), a portion of the synthesized extension product, or a portion of both. If the complementary DNA on the substrate is complementary to a portion of the primer, it would be necessary to remove nonhybridized, labeled primers from the reaction mixture prior to contact of the mixture with the substrate-bound oligonucleotides. Otherwise, the presence of label on the substrate might not be the result of a match between the labeled nucleotide and the test nucleotide, but might simply result from the presence of primer which failed to hybridize (except in the case of the nucleotide replacement assay). This could be accomplished in a variety of ways including: ensuring that most of the labeled primer molecules had an opportunity to hybridize to the test DNA and undergo reaction with the proofreading polymerase; treating the unreacted primers with a potent exo- or endonuclease preferring single stranded DNA to excise the labeled terminal nucleotide; or alternatively, removing unreacted primer molecules from the solution containing the extension product.

In order to ensure that most of the labeled primers participate in the reaction of the invention, it is helpful to repeat the primer annealing and exonucleolytic phases of the reaction many times. For example, the reaction may be heated to dissociate hybridized extended primer and test DNA and then cooled to permit annealing of new primers to the test DNA (PCR conditions). If the proofreading polymerase used is not heat stable, then more would be added, and the reaction mixture incubated under conditions to permit exonucleolytic action and polymerization. Alternatively, a strand displacing polymerase (e.g., phi-80 polymerase, which is commonly used in RCA) or a phage derived RNA polymerase (e.g., T7 polymerase) can be employed to generate new single stranded copies of the template in an isothermal reaction.

Instead of ensuring that most of the primers participate in the reaction, all of the labeled unreacted primers could be removed from the system by, for example, using a potent single-strand specific DNA exonuclease such as E. coli Exonuclease I, or a single strand specific endonuclease such as Mung Bean nuclease or SI nuclease. By this approach, the nuclease is added to the mixture after completion of the reaction with the proofreading polymerase of the invention. After a sufficient time of incubation, the unreacted single-stranded primers will be degraded to mononucleotides (and the label will be liberated as a labeled mononucleotide), but little or none double stranded extension products will be affected.

Another approach for eliminating unreacted, labeled primers as a potential source of unwanted background is to provide modified nucleoside triphosphates for incorporation into the extension product, which triphosphates when incorporated into this extension product facilitate separation of the exterior product from unreacted primer. For example, the nucleoside triphosphates may be modified with biotin, and then this biotinylated extension product could readily be identified and/or separated from unreacted primer. Alternatively, the extension product could be treated with a backstrand (reverse) primer including a biotin moiety or a tail, the backstrand primer capable of hybridizing with the extension product (as in PCR). The resulting double stranded DNA then could be separated from unreacted, labeled primer via the biotin moiety or by hybridization to the tail, and the presence or absence of label or the extension product determined. Standard purification techniques that remove single stranded DNA (e.g., QIAquick membranes, or hydroxyapatite chromatography), or which separate small molecules from larger ones (e.g., dialysis or gel filtration) could also be used. The extension product may be amplified, e.g., by PCR or RCA, prior to capture using the tails of the invention.

Applications of the Methods of the Invention

The methods of the invention may be used in connection with many types of medical tests, including gene typing, karyotyping, genotyping, DNA family planning, prenatal testing, diagnostics (including infectious disease), pharmacogenetics, toxicology, paternal determination, and forensic analysis. It is particularly useful in determining an individual's genotype at the test locus, especially as the genotype relates to the existence of an allele or mutation responsible for a disease state, therapeutic response, or as it relates to an individual's identity.

In one embodiment, the methods of the invention may be used advantageously to determine allelic variation in genotyping studies. For example, if allelic variation is due to a single nucleotide substitution (or is correlated with such a substitution), then test DNA can be treated using primers to both alleles to determine whether an individual is homozygous or heterozygous with respect to those alleles. By use of the primers with unique tails described in one embodiment of this invention, it is possible to test for all of the alleles of a single locus or even all of the alleles of several loci in a single reaction.

The invention may be employed to detect allelic variation or polymorphism due to a single base substitution on a strand of DNA. Such single nucleotide variation is known to be responsible for particular disease states, including beta-thalassemia, hemophilia, sickle cell anemia, and cystic fibrosis, and many others. When associated with restriction endonuclease cleavage sites, such variation results in restriction fragment length polymorphism (RFLP; Lench et al., The Lancet, Jun. 18, 1988, pp. 1356-1358). More generally, such variants are referred to as single nucleotide polymorphisms (SNPs).

A particularly useful application of the methods of the invention is in the field of pharmacogenomics. As described above, using the methods of the present invention a high resolution map can be generated from a combination or subset of some ten-million estimated SNPs in the human genome. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

This invention is further illustrated by the following examples which should not be construed as limiting. The contents of all references, patents and published patent applications cited throughout this application including the Figures and the Sequence Listing are incorporated herein by reference.

### EXAMPLES

### EXAMPLE 1

### ONE-COLOR EXO-PROOFREADING ASSAY FOR THE DETECTION OF SINGLE NUCLEOTIDE POLYMORPHISM (SNP) AT POSITION 341 IN THE NAT-2 GENE USING FLUORESCENTLY LABELED PRIMERS

The Exo-proofreading PCR was performed using either the PWO Polymerase kit from Boehringer Mannheim (Cat # 1644 947) or the Native Pfu Polymerase from Stratagene (cat # 600135) and the following primer sequences designed to detect a single nucleotide polymorphism at position 341 of the human NAT2 gene: Forward 5′ CTTCTCCTGCAGGTGACCAT*-3′ (SEQ ID NO:5, where * represents the fluorescein label that was used). Forward 5′ CTTCTCCTGCAGGTGACCAT*X- 3′ (SEQ ID NO:6, Penultimate base label); 5′ CTGAGGGCTGATCCTTCCCAG-3′ (SEQ ID NO:7, reverse PCR primer).

The total reaction volume was 50 μl, and contained 600 nM of each primer, 200 μM dNTPs, 10 mM Tris, pH8.85, 25 mM KCL, 50 mM (NH₄) 2SO₄ and 20 mM MgSO₄, and 2 ng of diluted primary NAT-2 PCR product generated from individuals homozygous for either the NAT2-T³⁴¹ or the NAT2-C³⁴¹ allele (as determined by DNA sequencing). Cycling conditions were as follows: 2 minutes at 94° C., followed by 35 cycles of [15 seconds at 94° C., 30 seconds at 60° C. [MRMI]and 35 seconds at 72° C.], with a final extension of 5 minutes at 72° C. The resulting PCR products were analyzed on a 12 cm 4.5% Acrylamide gel using an ABI 377 sequencer.

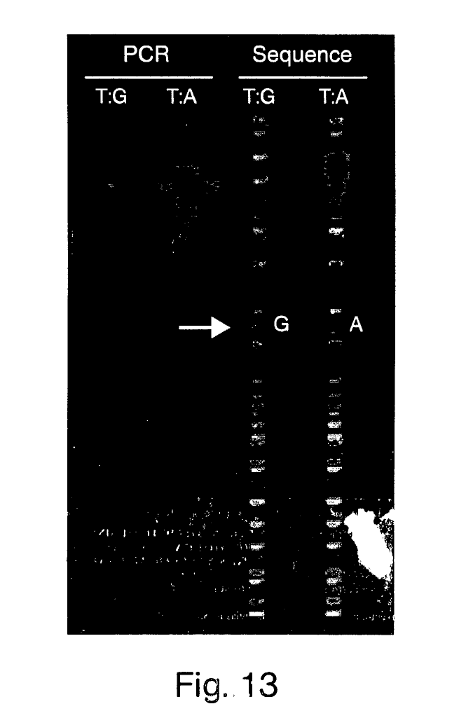

As indicated in FIG. 13, a signal is observed with the NAT2-T³⁴¹ template (T:A match with the labeled primer) but not the NAT2-C³⁴¹ template (T:G mismatch with the labeled primer). The PCR products were sequenced to verify the identity of the variable nucleotide using the reverse PCR primer and a Big Dye terminator sequencing kit (PE/Applied Biosystems Corporation).

### EXAMPLE 2

### TWO-COLOR EXO-PROOFREADING ASSAY USING PFU POLYMERASE ON ASM 698₁₃ B_{—}1

Samples containing a genomic PCR product were diluted 1:1000 using autoclaved RODI water, and 1.3 μl of diluted sample was transferred to a 96 well plate (Marsh BioMedical Products, Rochester, N.Y.; Cat. No. AB0800 PCR Plate-AB-1000/150/B). The following reagents were added to each sample: 0.5 μl of 10 mM dNTP stock (Roche, Nutley, N.J.; Cat. No. PWO 1644955 and 1644947, TAW), 2.5 μl of 10X PCR Buffer A:600 mM Tris-HCl, pH 8.5, 150 mM (NH₄)₂ SO_{4,}15 mM MgCl_{2,} 2 μl of 7.5 μM ASM73-3′ C (GAGCATGGCAGGCCCTGGC* SEQ ID NO:8) primer labeled with FAM fluorescent dye, 2μl of 7.5μM ASM75-3′ T (AATGAGCATGGCAGGCCCTGGT* SEQ ID NO:9) primer labeled with ROX fluorescent dye, 2 μl of 7.5 μM ASM74 (TCGAGGCATTTGCCCTGAACA SEQ ID NO: 10), 0.45 μl of Cloned Pfu DNA Polymerase 2.5U/μl (Stratagene La Jolla, Calif.; Cat. No. 600159 P+U 600159) and 14.25 μl of autoclaved RODI water. The plate was vortexed, centrifuged at 1000 r.p.m. (Beckman, Fullerton, Calif.; GS 6R Centrifuge), and covered with “Sealplate” adhesive polyester film (Marsh BioMedical Products; Cat. No. SP100-S). The plate was then thermocycled (NIJ tetrad; MJ Research, Waltham, Mass.) using the following program: [a. 94° C. 1 minute; b. 94° C. 10 seconds; c. 65° C. 35 seconds; d. 72° C. 30 seconds; e. Repeat steps b-d 34 times; f. 72° C. 5 min; g. Store at 4° C. until plate is removed].

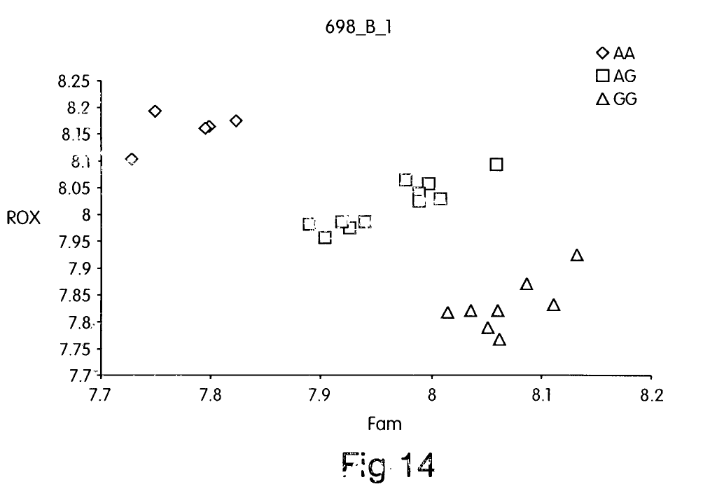

After thermocycling the unincorporated primers and cleaved tags were removed using a QIAquick 96 PCR purification kit (Qiagen, Valencia, Calif.; Cat. No.28181) according to the manufacturer's directions. Samples were analyzed on an Analyst HT Fluorescence Plate Reader (LJL Biosystems, now Molecular Devices, Sunnyvale, Calif.) by the following method. 25 μl of cleaned sample was transferred into HE Microplate (LJL Biosystems; Cat. No. 42-011) and the fluorescence of the ROX and FAM labels were measured at the following wavelengths: ROX: Excitation 580 Emission 610, FAM: Excitation 490 Emission 520. The results of the assay on 24 samples are shown in FIG. 14.

### EXAMPLE 3

### EXO-PROOFREADING PCR USING PWO POLYMERASE ON GTC216Q+1 USING GENOMIC DNA

Samples containing genomic DNA were diluted to a concentration of 4.5ng/μl, and 5μl was transferred to a 96-well plate (Marsh BioMedical Products Cat # AB0800). The following reagents were added to each sample: 1 μl of 10 mM dNTP stock (Roche Cat #), 5μl of 10X PWO DNA Polymerase Buffer w/o MgSO_{4:} 100 mM Tris-HCl, pH 8.85, 250 mM KC1, 50 mM (NH₄)₂SO₄ (Roche Cat#:1644955), 3 μl of 25 mM MgSO₄, 2μl of 7.5μM ASM39-3′ T ( NNNNNNNNNNNNG* ) primer labeled with TAMRA fluorescent dye, 2μl of 7.5μM A47-3′ T (NNNNNNNN NNC*) primer labeled with FAM fluorescent dye, 2μl of 7.5μM A30, 2μl of 7.5μM A48, 0.25μl of Pwo Polymerase 5U/μl (Roche Cat # 1644955) and 32.25μl of nuclease free water. The plate was vortexed, centrifuged at 1000 r.p.m. (Beckman GS 6 R Centrifuge), and covered with ‘sealplate’ adhesive polyester film (Marsh BioMedical Products Cat # SP100-S). The plate was then thermocycled (MJ tetrad; MJ Research) using the following program: a. 94° C. 1 minute; b. 94° C. 10 seconds; c. 60° C. 35 seconds; d. 72° C. 30 seconds; e. Repeat steps b-d 55 times; f. 72° C. 5 min; g. Store at 4° C. until plate is removed.

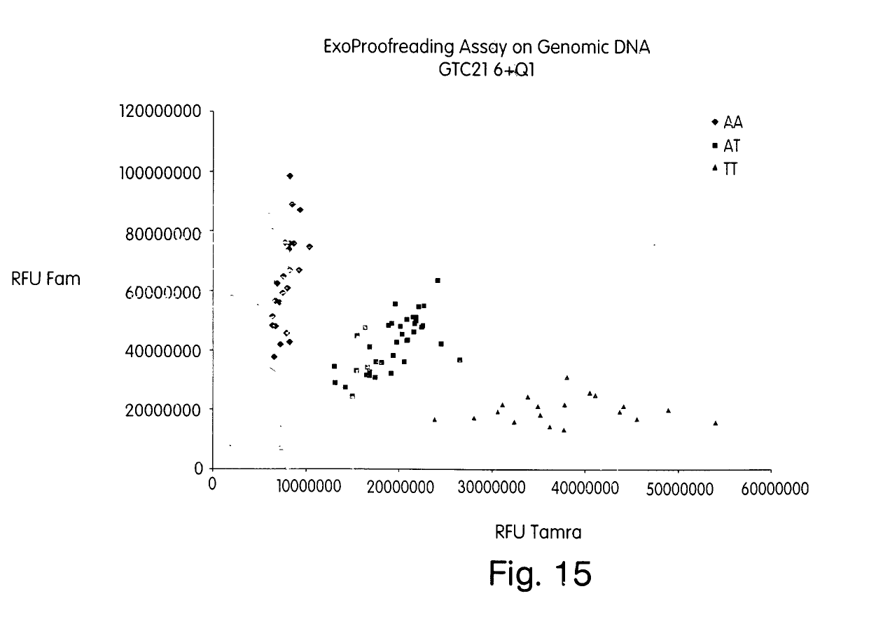

After thermocycling the unincorporated primers and cleaved tags were removed using a QIAquick 96 PCR purification kit (Qiagen Cat# 28181) according to the manufactures directions. Samples were analysed on an Analyst HT Fluorescence Plate Reader (LJL Biosystems) by the following method. 25 μl of cleaned sample was transferred into HE Microplate (LJL Biosystems Cat# 42-011) and the fluorescence of the TAMRA and FAM labels were measured at the following wavelengths: TAMRA: Excitation 550 Emission 580, FAM: Excitation 490 Emission 520. The results of this assay on 96 samples are shown in FIG. 15.

### EXAMPLE 4

### EXO-PROOFREADING PCR WITH INTEGRATED DIALYSIS AND DETECTION OF EXCISED NUCLEOTIDE PRODUCTS

The Exo-proofreading PCR was performed using either the Pwo Polymerase kit from Boehringer Mannheim (Cat # 1644 947) and the following primer sequences designed to detect a single nucleotide polymorphism at position 341 of the human NAT2 gene: 5′ CTTCTCCTGCAGGTGACCAT*- 3′(SEQ ID NO:5, where * represents the fluorescein label that was used), and 5′ CTGAGGGCTGATCCTTCCCAG-3′(SEQ ID NO:7, reverse PCR primer). The reaction was carried out in a volume of 50 μl, and contained 600 nM primers, 200 μM dNTPs, 10 mM Tris, pH 8.85, 25 mM KCL, 50mM (NH₄)2 S_{4,} 20 mM MgSO₄, and 2 ng of diluted primary NAT-2 PCR product generated from individuals homozygous for either the NAT2-T³⁴¹ or the NAT2-C³⁴¹ allele. Cycling conditions were as follows: 2 minutes at 94° C., followed by 35 cycles of [15 seconds at 94° C., 30 seconds at 60° C. and 35 seconds at 72° C.], with a final extension of 5 minutes at 72° C.

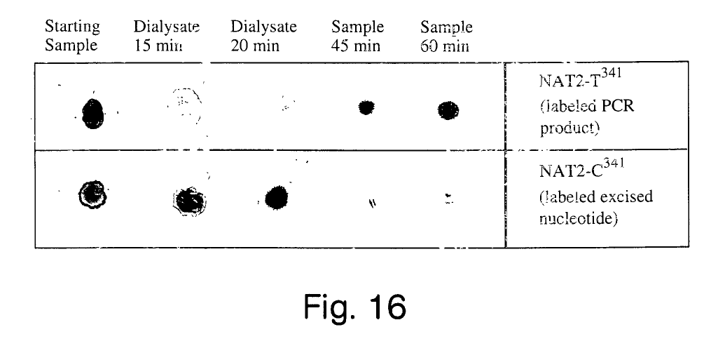

After PCR, 5 μl of the NAT2-T³⁴¹ sample was placed in a dialysis device, and 5 μl of the NAT2-C³⁴¹ sample was placed into another dialysis system. Both devices contained Spectrum Brand 100k MWCO CE Dialysis membranes, and 5μl of distilled water in a chamber on the opposite side of the membrane from the sample (dialysate). In one experimental run, the dialysate from each sample was retrieved 15 min and placed onto a glass slide; the remaining sample was retrieved after 45 minutes, and also placed on the slide. In a second experimental run, the dialysate from each sample was retrieved 20 min and placed onto the slide; the remaining sample was retrieved after 60 minutes, and also placed on the slide. The glass slide was then read with a Fuji Fluorescent reader. The results are shown in FIG. 16.

The top row of FIG. 14 shows the results from the NAT2-T³⁴¹ sample. The bottom row shows the results from the NAT2-C³⁴¹ sample. The first column shows the initial samples before any dialysis has taken place, indicating that labeled constituents were present in both samples. The second and third columns show the dialysate after 15 and 20 min of dialysis, respectively; the label in the NAT2-T³⁴¹ sample is associated with the starting primers and the PCR extension product and therefore does not dialyze rapidly across the membrane; the label in the NAT2-T³⁴¹ sample is associated with the starting primers and the excised nucleotide, due to the presence of a mismatch, and therefore diffuses rapidly across the membrane from the sample into the dialysis solution. The forth and fifth columns show the remaining samples after dialysis for 45 and 60 minutes, which is a sufficient time period for dialysis of the starting primers of the invention to occur, but not the PCR extension products. Thus, the label associated 341 with the extension products in the NAT2-T sample is still visible, while the label associated with the remaining primer (and nucleotide excision product) in the NAT2-C³⁴¹ sample has substantially dialyzed out of the sample.

### EXAMPLE 5

### TWO-COLOR EXO-PROOFREADING ASSAY USING PWO POLYMERASE FOR THE DETECTION OF SINGLE NUCLEOTIDE POLYMORPHISM (SNP) ON GTC216 ₁₃ Q_{13 +}1 USING FLUOROSCENCE POLARIZATION DETECTION METHOD

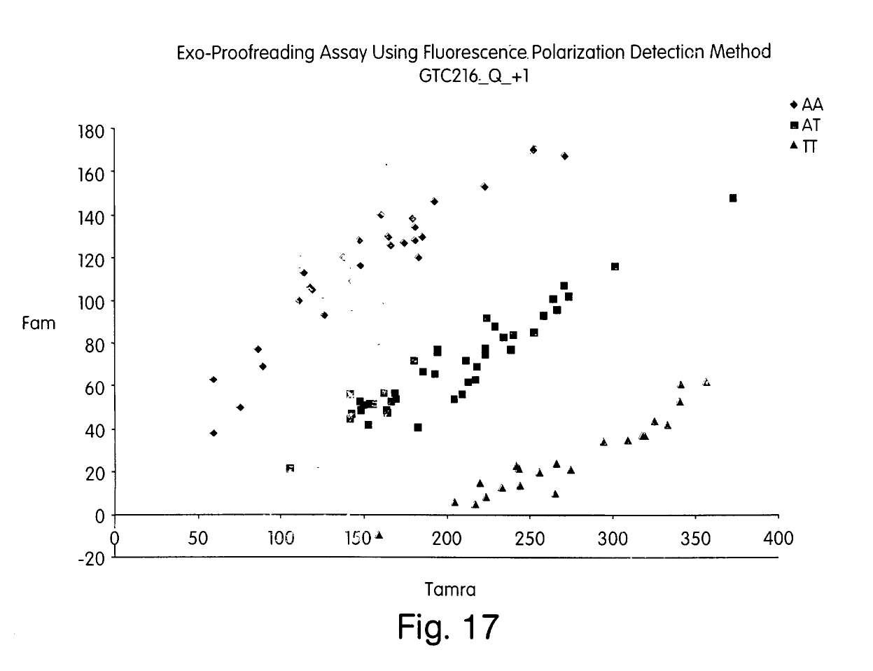

Samples containing a genomic PCR product were diluted 1:1000 using nuclease free water (Promega Part # P119C), and 2.5μl of diluted sample was transferred to a 96-well plate (Marsh BioMedical Products Cat # AB0800). The following reagents were added to each sample: 1 μl of 10 mM dNTP stock (Roche Cat #), 5μl of 10 X PWO DNA Polymerase Buffer w/o MgSO4: 100 mM Tris-HCl, pH 8.85, 250mM KCl50mM (NH₄)₂SO₄ (Roche Cat#: 1644955), 3μl of 25mM MgSO_{4,} 2μl of 7.5μM ASM29-3′ T (NNNNNNNNNNNNT*) primer labeled with FAM fluorescent dye, 2μl of 7.5μM ASM47-3′ T (NNNNNNNNNNNNT*) primer labeled with TAMRA fluorescent dye, 2μl of 7.5μM ASM30, 2μl of 7.5μM ASM48, 0.25μl of Pwo Polymerase 5U/μl (Roche Cat # 1644955) and 32.25μl of nuclease free water. The plate was vortexed, centrifuged at 1000 r.p.m. (Beckman GS 6R Centrifuge), and covered with ‘sealplate’ adhesive polyester film (Marsh BioMedical Products Cat # SP100-S). The plate was then thermocycled (MJ tetrad; MJ Research) using the following program: a. 94° C. 1 minute; b. 94° C. 10 seconds; c. 60° C. 35 seconds; d. 72° C. 30 seconds; e. Repeat steps b-d 54 times; Hold at 4° C. until plate is removed. Samples were analysed on an Analyst HT Fluorescence Plate Reader (LJL Biosystems) by the following method: 2μl of sample was added to 22μl of 1 N NaOH in an HE Microplate (LJL Biosystems Cat# 42-011), and fluorescence polarization measurments of the TAMRA and FAM labels were taken at the following wavelengths: TAMRA: Excitation 550 Emission 580, FAM: Excitation 490 Emission 520. The results of this assay on 96 samples are shown in FIG. 17.

### EXAMPLE 6

### USE OF ELECTROPHORE MASS-TAGS AND LI-EC-TOF-MS DETECTION WITH THE PROOFREADING PCR ASSAY

The Exo-proofreading PCR was performed using either the PWO Polymerase kit from Boehringer Mannheim (Cat # 1644 947) and the following primer sequences designed to detect a single nucleotide polymorphism at position 341 of the human NAT2 gene: 5′ CTTCTCCTGCAGGTGACCAT*- 3′(SEQ ID NO:5, where * represents the methyl {2,3,5,6-tetrafluoro-4-[4′-(2“-oxobutanephenoxy)-2′,3′,5′,6′-(tetraflourofluorophenyl)phenoxy]}isonipecotate NHS ester label that was used). 5′ CTGAGGGCTGATCCTTCCCAG-3′(SEQ ID NO:7, reverse PCR primer).

The total reaction volume was 50μl, and contained 600 nM of each primer, 200 μM dNTPs, 10 mM Tris, pH8.85, 25 mM KCL, 50mM (NH₁₄)2SO₄ and 20 mM MgSO₄, and 2 ng of diluted primary NAT-2 PCR product generated from individuals homozygous for either the NAT2-T³⁴¹ or the NAT2-C³⁴¹ allele (as determined by DNA sequencing). Cycling conditions were as follows: 2 minutes at 94° C., followed by 35 cycles of [15 seconds at 94° C., 30 seconds at 60° C. and 35 seconds at 72° C.], with a final extension of 5 minutes at 72° C. The resulting PCR extension products were purified on a QIAquick membrane using the procedure recommended by the manufacturer (QIAGEN), and spotted on a silver-coated sample plate for a Bruker BIFLEX III TOF instrument. The samples were analyzed in the BIFLEX instrument after illumination with a defocused N₂ laser.

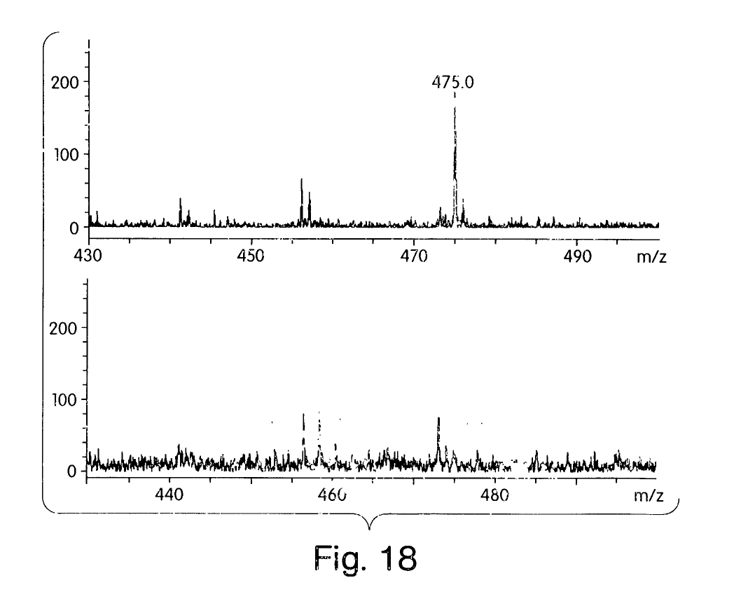

As indicated in FIG. 18, a strong signal was observed with the expected mass of the electrophore tag (475 Daltons) in the sample derived from the NAT2-T³⁴¹ template (match to the primer sequence) but little or no signal was observed with the NAT2-C³⁴¹ template (mismatch with the primer).

### EXAMPLE 7

### TWO-COLOR EXO-PROOFREADING ASSAY FOR ALLELE FREQUENCY DETERMINATION FOR ASM 454 _F_-2

Samples containing a genomic PCR product were diluted 1:1000 using autoclaved RODI water. Thirteen samples containing genotype homozygous C were pooled. Also, thirteen samples with genotype homozygous T were pooled. Known mixtures of the two genotypes were made ranging from 100% C 0% T to 0% C 100% T. 1.3 μl of each mixture was transferred to a 96 well plate (Marsh BioMedical Products Cat# AB0800). Each genotype mix was run 6 times. The following reagents were added to each sample: 0.5 μl of 10 mM dNTP stock (Roche Cat#), 2.5 μl of PWO DNA Polymerasc 10 X Buffer w/o MgSO4: 100 mM Tris-HCl, pH 8.85,50 mM (NH₄)₂SO_{4,} 250 mM KCI (Roche Cat#: 1644955) 1.5 μl of 25mM MgSO₄ (Roche Cat#1644955), 2 μl of 7.5 μM ASM164-3′ C (CATGGGCTCCCTCGGTLC* SEQ ID NO:11) primer labeled with FAM fluorescent dye, 2μl of 7.5 μM ASM166-3′ T (CATGGGCTCCCTCGGTT* SEQ NO:12) primer labeled with ROX fluorescent dye, 2 μl of 7.5 μM ASM165 (CCGGGGAAGTCGATATTGTT SEQ ID NO:13), 0.125 of PWO DNA Polymerase 5U/μl (Roche Cat# 1 644 955) and 13.07 μl of autoclaved RODI water. The plate was vortexed, centrifuged at 1000 rpm (Beckman GS 6R Centrifuge), and covered with “Sealplate” adhesive polyester film (Marsh BioMedical Products Cat# SP100-S). The plate was then thermocycled (MJ tetrad; MJ Research) using the following program: [a. 94° C. 1 minute; b. 94° C. 10 seconds; c. 62° C. 35 seconds; d. 72° C. 30 seconds; e. Repeat steps b-d 34 times; f. 72° C. 5 min; g. Store at 4° C. until plate is removed].

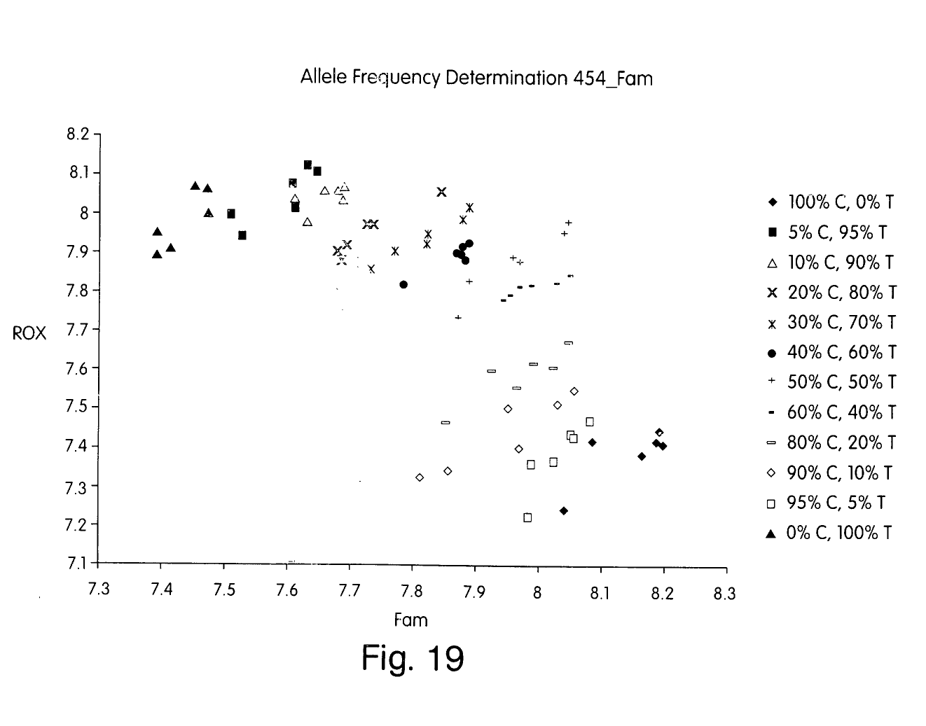

After thermocycling the unincorporated primers and cleaved tags were removed using a QIAquick 96 PCR purification kit (Qiagen Cat#28181) according to the manufacturer's directions. Samples were analyzed on an Analyst HT Fluorescence Plate Reader (LJL Biosystems) by the following method. 25 μl of cleaned sample was transferred into HE Microplate (LJL Biosystems Cat# 42-011) and the fluorescence of the ROX and FAM labels were measured at the following wavelengths: ROX: Excitation 580 Emission 610, FAM: Excitation 490 Emission 520. The results of the assay are shown in FIG. 19. Based on a simple inspection of the graph, the relative frequency of each base within the pooled set of samples was determined.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for detecting the presence or absence of a first nucleotide, at a position within a strand of DNA in a sample, comprising:
forming an admixture of a primer and said strand of DNA in said sample and imposing hybridization conditions on said primer and said DNA strand to form a hybridization product, said primer comprising a sequence of DNA which hybridizes with said strand of DNA adjacent to said first nucleotide position and having a second nucleotide opposite said first nucleotide position, said second nucleotide associated with a detectable label, said second nucleotide hybridizing to said first nucleotide in the event said second nucleotide is complementary to said first nucleotide and said second nucleotide not hybridizing to said first nucleotide in the event of said second nucleotide is not complementary;
applying a proofreading polymerase to the hybridization product under conditions in which said second nucleotide is preferentially excised to form a fluorescently labeled nucleotide product in the event said second nucleotide is not hybridized to said first nucleotide, and in which said second nucleotide is preferentially incorporated into an extension product in the event said second nucleotide is hybridized to said first nucleotide;
monitoring said sample for the presence of a fluorescent label in association with at least one of said fluorescently labeled nucleotide excision product, said extension product, or said primer using fluorescent polarization, which fluorescent label associated with an excess of said nucleotide excision product is indicative of the absence of said first nucleotide, and which fluorescent label associated with an excess of said extension product is indicative of the presence of said first nucleotide.

2. A method for detecting the presence or absence of a first nucleotide, at a position within a strand of DNA in a sample, comprising:
forming an admixture of a primer and said strand of DNA in said sample and imposing hybridization conditions on said primer and said DNA strand to form a hybridization product, said primer comprising a sequence of DNA which hybridizes with said strand of DNA adjacent to said first nucleotide position and having a second nucleotide opposite said first nucleotide position, said second nucleotide associated with a mass-tag, said second nucleotide hybridizing to said first nucleotide in the event said second nucleotide is complementary to said first nucleotide and said second nucleotide not hybridizing to said first nucleotide in the event of said second nucleotide is not complementary;
applying a proofreading polymerase to the hybridization product under conditions in which said second nucleotide is preferentially excised to form a nucleotide excision product attached to a mass-tag in the event said second nucleotide is not hybridized to said first nucleotide, and in which said second nucleotide is preferentially incorporated into an extension product in the event said second nucleotide is hybridized to said first nucleotide;
monitoring said sample for the presence of a mass-tag in association with at least one of said nucleotide excision product, said extension product, or said primer using mass spectrometry, which mass-tag associated with said nucleotide excision product in concentrations greater than background is indicative of the absence of said first nucleotide, and which mass-tag associated with said extension product in concentrations greater than background is indicative of the presence of said first nucleotide.

3. A method for detecting the presence or absence of a first nucleotide, at a position within a strand of DNA in a sample, comprising:
forming an admixture of a primer and said strand of DNA in said sample and imposing hybridization conditions on said primer and said DNA strand to form a hybridization product, said primer comprising a sequence of DNA which hybridizes with said strand of DNA adjacent to said first nucleotide position and having a second nucleotide opposite said first nucleotide position, said second nucleotide associated with a label, said second nucleotide hybridizing to said first nucleotide in the event said second nucleotide is complementary to said first nucleotide and said second nucleotide not hybridizing to said first nucleotide in the event said second nucleotide is not complementary;
applying a proofreading polymerase to the hybridization product under conditions in which said second nucleotide is preferentially excised to form a labeled nucleotide product in the event said second nucleotide is not hybridized to said first nucleotide, and in which said second nucleotide is preferentially incorporated into an extension product in the event said second nucleotide is hybridized to said first nucleotide;
providing a dialysis chamber having a dialysis membrane with a molecular weight cut-off such that the labeled nucleotide excision product, the primer, and the extension product may pass through at substantially different rates;
providing a means for introducing the admixture into a chamber on a first side of the dialysis membrane, and for introducing a dialysis solution into a chamber on a second side of the dialysis membrane opposite the first side of the dialysis membrane;
monitoring the sample on the first side of the dialysis membrane, or monitoring the dialysis solution on the second side of the dialysis membrane, or both, for the presence of a label;
the presence of a label in the dialysis solution in concentrations greater than background after a short time is indicative of the absence of the first nucleotide, and the presence of a label remaining in the sample chamber in concentrations greater that background after a long time is indicative of the presence of the first nucleotide.

4. A method for detecting the presence or absence of a first nucleotide, at a position within a strand of DNA in a sample, comprising:
forming an admixture of a primer, said strand of DNA in said sample, and a mixture of labeled dideoxynucleotides, said primer comprising a sequence of DNA which hybridizes with said strand of DNA adjacent to said first nucleotide position and having a second nucleotide opposite said first nucleotide position which is not complementary to said first nucleotide;
imposing hybridization conditions on said primer and said DNA strand to form a hybridization product;
applying a proofreading polymerase to the hybridization product under conditions in which said second nucleotide is excised and a labeled dideoxynucleotide is inserted that is complementary to said first nucleotide position; and
monitoring said sample for the presence of a label in association with at least one of said primer or said hybridization product, which label associated with said primer or said hybridization product in concentrations greater than background is indicative of the presence of said first nucleotide.

5. A method for detecting the presence or absence of a first nucleotide, at a position within a strand of DNA in a sample, comprising:
forming an admixture of a primer and a strand of DNA in said sample and imposing conditions such that a hybridization product is formed between the primer and said DNA strand, said primer comprising a sequence of DNA which hybridizes with the strand of DNA and having a second nucleotide containing a fluorescent label opposite the position of the first nucleotide and said primer also containing a quencher moiety attached at a position adjacent to the second nucleotide, the second nucleotide hybridizing to the first nucleotide in the event that the second nucleotide is complementary to the first nucleotide and the second nucleotide not hybridizing to the first nucleotide in the event that the second nucleotide is not complementary;
applying a proofreading polymerase to the hybridization product under conditions in which the second nucleotide containing the fluorophore is preferentially excised in the event that the second nucleotide is not hybridized to the first nucleotide and in which the second nucleotide containing the fluorophore is preferentially incorporated into primer extension product in the event that the second nucleotide is hybridized to the first nucleotide;
monitoring the sample for emission from the fluorophore, the presence of fluorescence emission at levels greater than background being indicative of the absence of the first nucleotide, and the absence of fluorescence emission being indicative of the presence of the first nucleotide.

6. The method of claim 5, wherein said quencher moiety is attached about 1-10 nucleotides away from the position of the fluorescent label on said second nucleotide opposite said first nucleotide when said primer and said DNA sample are paired.

7. The method of claim 5, wherein said fluorophore is a fluorescent label having an absorption maximum in a wavelength range between 340 nm and 820 nm and an emission maximum in a wavelength range between 370 nm and 850 nm.

8. The method of claim 5, wherein said fluorophore is a fluorescent label selected from the group consisting of: Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680, AMCA, AMCA-S, BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, Carboxyrhodamine 6 G, carboxy-X-rhodamine (ROX), Cascade Blue, Cascade Yellow, Cy3, Cy5, Cy3.5, Cy5.5, Dansyl, Dapoxyl, Dialkylaminocoumarin, 4 ′,5 ′-Dichloro-2 ′,7′-dimethoxy-fluorescein, DM-NERF, Eosin, Erythrosin, Fluorescein, FAM, Hydroxycoumarin, IRD40, IRD 700, IRD 800, JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodarnine (TMR), Carboxytetramethylrhodamine (TAMRA), Texas Red, and Texas Red-X.

9. The method of claim 5, wherein said quencher moiety displays a Forster radius of greater than 30 ÅAngstroms when paired with a fluorophore under conditions where FRET may occur, and having a broad absoption spectrum with an absorption maximum in the range of 480-700 nm.

10. The method of claim 5, wherein said quencher moiety is selected from the group consisting of DABCYL, QSY-7, QSY-33, Q1, BHQ-1, BHQ-2, and BHQ-3.

11. A method for detecting the presence or absence of a first nucleotide, at a position within a strand of DNA in a sample, comprising:
forming an admixture of a primer and a strand of DNA in said sample and imposing conditions such that a hybridization product is formed between the primer and said DNA strand, said primer comprising a sequence of DNA which hybridizes with the strand of DNA and having a second nucleotide containing a quencher opposite the position of the first nucleotide and said primer also containing a fluorescent label attached at a position adjacent to said second nucleotide, the second nucleotide hybridizing to the first nucleotide in the event that the second nucleotide is complementary to the first nucleotide and the second nucleotide not hybridizing to the first nucleotide in the event that the second nucleotide is not complementary;
applying a proofreading polymerase to the hybridization product under conditions in which the second nucleotide containing the quencher is preferentially excised in the event that the second nucleotide is not hybridized to the first nucleotide and in which the second nucleotide containing the quencher is preferentially incorporated into primer extension product in the event that the second nucleotide is hybridized to the first nucleotide;
monitoring the sample for emission from the fluorophore, the presence of fluorescence emission at levels greater than background being indicative of the absence of the first nucleotide, and the absence of fluorescence emission being indicative of the presence of the first nucleotide.

12. The method of claim 11, wherein said quencher moiety is attached about 1-10 nucleotides away from the position of the fluorescent label on said second nucleotide opposite said first nucleotide when said primer and said DNA sample are paired.

13. The method of claim 11, wherein said fluorophore is a fluorescent label having an absorption maximum in a wavelength range between 340 nm and 800 nm and an emission maximum in a wavelength range between 400 nm and 850 nm.

14. The method of claim 11, wherein said fluorophore is a fluorescent label selected from the group consisting of: Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680, AMCA, AMCA-S, BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, Carboxyrhodamine 6G, carboxy-X-rhodamine (ROX), Cascade Blue, Cascade Yellow, Cy3, Cy5, Cy3.5, Cy5.5, Dansyl, Dapoxyl, Dialkylaminocoumarin, 4 ′,5 ′-Dichloro-2 ′,7′-dimethoxy-fluorescein, DM-NERF, Eosin, Erythrosin, Fluorescein, FAM, Hydroxycoumarin, IRD40, IRD 700, IRD 800, JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

15. The method of claim 11, wherein said quencher moiety displays a Forster radius of greater than 30 Angstroms when paired with a fluorophore under conditions where FRET may occur, and having a broad absoption spectrum with an absorption maximum in the range of 480-700 nm.

16. The method of claim 5, wherein said quencher moiety is selected from the group consisting of DABCYL, QSY-7, QSY-33, Q1, BHQ-1, BHQ-2, and BHQ-3.

17. A method for detecting the presence or absence of a first nucleotide, at a position within a strand of DNA in a sample, comprising:
forming an admixture of a primer and a strand of DNA in said sample and imposing conditions such that a hybridization product is formed between the primer and said DNA strand, said primer comprising a sequence of DNA which hybridizes with the strand of DNA and having a second nucleotide containing an electrophoretic tag (e-tag) moiety opposite the position of the first nucleotide, the second nucleotide hybridizing to the first nucleotide in the event that the second nucleotide is complementary to the first nucleotide and the second nucleotide not hybridizing to the first nucleotide in the event that the second nucleotide is not complementary;
applying a proofreading polymerase to the hybridization product under conditions in which the second nucleotide containing the e-tag is preferentially excised in the event that the second nucleotide is not hybridized to the first nucleotide and in which the second nucleotide containing the e-tag is preferentially incorporated into primer extension product in the event that the second nucleotide is hybridized to the first nucleotide;
monitoring the sample for the presence of e-tag labeled nucleotide products by electrophoretic separation, the presence of such e-tag nucleotide products at levels greater than background being indicative of the absence of the first nucleotide, and the absence of such e-tag nucleotide products being indicative of the presence of the first nucleotide.

18. The method of any one of claims 1, 2, 3, 5, 11, or 17 wherein multiple nucleotide variants at the position of said first nucleotide are tested simultaneously in the same reaction vessel by using more than one labeled primer.

19. The method of any one of claims 1, 2, 3, 5, 11, or 17 wherein multiple nucleotide variants from different genetic loci are tested simultaneously in the same reaction vessel by using more than one labeled primer.

20. The method of any one of claims 1, 2, 3, 4,5, 11, or 17 wherein the amount of said nucleotide excision product and of said extension product are increased by means of an amplification reaction that results in faithful replication of said DNA strand in the sample.

21. The method of any one of claims 1, 2, 3, 5, 11, or 17 wherein the amount of said nucleotide excision product and of said extension product are increased by means of polymerase chain reaction (PCR) amplification in the presence of a reverse primer.

22. The method of any one of claims 1, 2, 3, 5, 11, or 17 wherein the amount of said nucleotide excision product and of said extension product are increased by means of a linear amplification reaction, such as a cycled hybridization and extension reaction.

23. The method of any one of claims 1, 2, 3, 5, 11, or 17 wherein the amount of said nucleotide excision product and said extension product is amplified by means of rolling circle amplification (RCA) after circularization of said DNA strand in the sample.

24. The method of any one of claims 1, 2, 3, 4, 5, 11, or 17 wherein said DNA of the sample is genomic DNA.

25. The method of any one of claims 1, 2, 3, 4, 5, 11, or 17 wherein said primer further comprises a tail that is non-complementary with said DNA strand.

26. The method of claim 2, wherein said mass-tag comprises an electrophore mass-tag and said mass spectrometry is electron-capture mass spectrometry.

27. The method of claim 2, wherein said mass-tag is a nucleotide or an oligonucleotide, and said mass spectrometry is matrix-assisted laser desorption/ionization time-of-flight mass spectrometry.

28. The method of claim 2, wherein said mass-tag is an organic molecule with a molecular weight between 100 and 2000 Daltons.

29. The method of claim 3, wherein said dialysis membrane comprises a semi-permeable microfiber.

30. The method of claim 3, wherein said dialysis occurs in a microfluidic channel in the absence of a semi-permeable membrane.

31. The method of claim 3, wherein said dialysis chamber has a molecular weight cut-off of about 100 KDa.

32. The method of claim 17, wherein said electrophoretic separation occurs in a polymer filled capillary or microchannel.

33. A method for determining allele frequency at a first nucleotide position within a strand of DNA in a sample, comprising:
providing a first primer, said first primer comprising a sequence of DNA which hybridizes with said strand of DNA adjacent to said first nucleotide position, and having a second nucleotide opposite said first nucleotide position, said second nucleotide associated with a first detectable label, said second nucleotide hybridizing to said first nucleotide in the event said second nucleotide is complementary to said first nucleotide and said second nucleotide not hybridizing to said first nucleotide in the event said second nucleotide is not complementary;
providing a second primer, said second primer comprising a sequence of DNA which hybridizes with said strand of DNA adjacent to said first nucleotide position, and having a third nucleotide opposite said first nucleotide position, said third nucleotide associated with a second detectable label, said third nucleotide hybridizing to said first nucleotide in the event said third nucleotide is complementary to said first nucleotide and said third nucleotide not hybridizing to said first nucleotide in the event said third nucleotide is not complementary;
forming an admixture of said first and second primers and said strand of DNA in said sample and imposing hybridization conditions on said first and second primers and said DNA strand to form a hybridization product;
applying a proofreading polymerase to the hybridization product under conditions in which said second and said third nucleotide is preferentially excised in the event said second and said third nucleotide is not hybridized to said first nucleotide, and in which said second and said third nucleotide is preferentially incorporated into an extension product in the event said second and said third nucleotide is hybridized to said first nucleotide;
monitoring said sample for the presence of a first or a second label in association with said extension product, wherein the ratio of said first and said second label is indicative of allele frequency at said first nucleotide position within a strand of DNA in a sample.
